(19) United States
(12) Patent Application Publication
(10) Pub. No.: US 2023/0001087 A1
(43) Pub. Date: Jan. 5, 2023

(54) MEDICAMENT DELIVERY DEVICE AND A DOSAGE MEASUREMENT SYSTEM

(71) Applicant: Sanofi, Paris (FR)

(72) Inventors: Martin Rebmann, Wil (CH); Roman Haettenschwiler, Wil (CH); Philipp Mueller, Wil (CH); Sven Zwicker, Wil (CH); Philipe Just, Wil (CH)

(21) Appl. No.: 17/777,331

(22) PCT Filed: Nov. 19, 2020
(86) PCT No.: PCT/EP2020/082610

§ 371 Date: May 17, 2022

(30) Foreign Application Priority Data
Nov. 21, 2019 (EP) 19306498.7

(51) Int. Cl. A61M 5/178

(52) U.S. Cl. CPC A61M 5/178; A61M 2205/581; A61M 5/31568

(57) ABSTRACT

The present disclosure relates to a medicament delivery device comprising: a reservoir for medicament; a dispensing mechanism operable to dispense medicament from the reservoir, the dispensing mechanism comprising a sleeve configured to rotate during the dispensing of medicament and having a plurality of formations at an end of the sleeve; and a dosage measurement system comprising at least one mechanically actuated sensor configured such that, in use, rotation of the sleeve causes successive formations to engage the sensor such that the sensor detects rotation of the sleeve and a processor configured to determine a dosage dispensed from the medicament reservoir based on the detected rotation of the sleeve.

### CROSS REFERENCE TO RELATED APPLICATIONS

The present application is the national stage entry of International Patent Application No. PCT/EP2020/082610, filed on Nov. 19, 2020, and claims priority to Application No. EP 19306498.7, filed on Nov. 21, 2019, the disclosures of which are incorporated herein by reference.

### TECHNICAL FIELD

The present disclosure relates to a medicament delivery device and a dosage measurement system for a medicament delivery device.

### BACKGROUND

A variety of diseases exists that require regular treatment by delivery of medicament using a medicament delivery device. Such delivery can be performed by using injection devices, which are applied either by medical personnel or by patients themselves. As an example, type-1 and type-2 diabetes can be treated by patients themselves by injection of insulin doses, for example once or several times per day. For instance, a pre-filled disposable insulin pen can be used as an injection device. Alternatively, a re-usable pen may be used. A re-usable pen allows replacement of an empty medicament cartridge by a new one. Either pen may come with a set of one-way needles that are replaced before each use. The insulin dose to be injected can then for instance be manually selected at the insulin pen by turning a dosage knob and observing the actual dose from a dose window or display of the insulin pen. The dose is then injected by inserting the needle into a suited skin portion and pressing an injection button of the insulin pen. To be able to monitor insulin injection, for instance to prevent false handling of the insulin pen or to keep track of the doses already applied, it is desirable to measure information related to a condition and/or use of the injection device, such as for instance information on the injected insulin dose.

### SUMMARY

The present disclosure relates to an improved medicament delivery device and a dosage measurement system for a medicament delivery device.

According to the present disclosure, there is provided a medicament delivery device comprising: a reservoir for medicament; a dispensing mechanism operable to dispense medicament from the reservoir, the dispensing mechanism comprising a sleeve configured to rotate during the dispensing of medicament and having a plurality of formations at an end of the sleeve; and, a dosage measurement system comprising at least one mechanically actuated sensor configured such that, in use, rotation of the sleeve causes successive formations to engage the sensor such that the sensor detects rotation of the sleeve and a processor configured to determine a dosage dispensed from the medicament reservoir based on the detected rotation of the sleeve.

The use of a mechanical sensor that engages with the sleeve provides a compact system for determining the dose dispensed from the medicament reservoir. A mechanical sensor is generally more energy efficient that other kinds of sensor, for example, a light gate, and thus a smaller battery can be used. Since the mechanical sensor engages formations on the sleeve of the dispensing mechanism, the system can be made more portable than if the sensor detected external components of the device.

In some embodiments, the plurality of formations comprise a plurality of teeth.

In some embodiments, the formations are formed on a proximal end of the sleeve.

The formations being on the proximal end of the sleeve allows for the sensor to be axially aligned with the sleeve. The sensor can be provided proximally of the sleeve.

In some embodiments, the sleeve is a dial sleeve or a drive sleeve.

In some embodiments, the sensor comprises a sensing member that is configured to move from an unactuated state to an actuated state when the sensing member is engaged by one of the formations during rotation of the sleeve, wherein the sensor detects movement of the sensing member between the unactuated and actuated states.

In some embodiments, each formation comprises a leading edge and wherein engagement of the sensor with the leading edge of a first of the formations until engagement of the sensor with the leading edge of an adjacent second of the formations during rotation of the sleeve represents one encoding period, and wherein the sensor is actuated for between 40 to 60% of the encoding period and, preferably, the sensor is actuated for about 50% of the encoding period. Such a configuration results in a symmetric scheme, which may be helpful for tolerance robustness as tolerances are in many situations symmetrically distributed.

In some embodiments, the medicament delivery device further comprises a dose dial and a housing, wherein the dose dial is configured to be rotated relative to the housing to set a dose of medicament to be delivered by the dispensing mechanism, and wherein the sensor is mounted to the dose dial.

The sensor being mounted to the dose dial allows for a compact medicament delivery device. In some embodiments, the sensor is mounted to the inside of the dose dial such that the dosage measurement system is discreet and compact.

In some embodiments, the medicament delivery device comprises a torque limiter, wherein the sensor is mounted to the dose dial via the torque limiter such that rotation of the dose dial relative to the housing with a torque greater than a predetermined limit causes the torque limiter to move to an open state such that the dose dial can rotate relative to the sensor and, preferably, the torque limiter comprises an overload clutch.

The torque limiter prevents damage to the sensor if the user exerts a large torque on the dose dial during the setting of a dose to be delivered by the measurement delivery device.

In some embodiments, the medicament delivery device further comprises a one-way mechanism, wherein the sensor is mounted to the dose dial via the one-way mechanism such that the sensor is resisted from rotating relative to the dose dial in the direction that the sleeve rotates during the dispensing of medicament and is permitted to rotate relative to the dose dial in the opposite direction that the sleeve rotates during the dispensing of medicament.

The one-way mechanism prevents damage to the sensor if the user urges the sleeve to rotate in a opposite direction to the direction that the sleeve is intended to rotate during the dispensing of medicament, since the sensor will rotate together with the sleeve in said opposite direction.

According to another aspect, there is provided a dosage measurement system for a medicament delivery device, wherein the medicament delivery device comprises: a housing containing a reservoir for medicament; and, a dispensing mechanism operable to dispense medicament from the reservoir and comprising a component configured to rotate during the dispensing of medicament, the component comprising a plurality of formations; and, an actuator configured to be movable relative to the housing upon actuation to operate the dispensing mechanism to dispense medicament from the reservoir, the dosage measurement system comprising: a sensor moveable from an idle position to a detecting position wherein rotation of the component causes successive formations to be detected by the sensor such that the sensor detects rotation of the component; a processor configured to determine a dosage dispensed from the medicament reservoir based on the detected rotation of the component; and, an activation switch moveable from an initial off state to an on state to activate the dosage measurement system upon actuation of the actuator, the sensor configured to be mounted to the actuator such that actuation of the actuator causes the sensor to move relative to the housing to reach the detecting position, wherein the activation switch reaches the on state prior to the sensor reaching the detecting position.

The configuration of the sensor and activation switch ensures that the dosage measurement system is activated prior to the sensor reaching the detecting position. This helps to ensure that the sensor is not in the detecting position before the dosage measurement system is powered on.

In some embodiments, the actuator is slidable relative to the housing. A sliding movement of the actuator may be easier for a patient, particularly if the patient is elderly or infirm.

In some embodiments, the activation switch comprises a pivotal member that pivots from the off state to the on state to activate the dosage measurement system.

In some embodiments, the medicament delivery device comprises a stop and wherein the activation switch is configured to rest against the stop when the activation switch is in the off state.

In some embodiments, the activation switch is configured to engage a part of the medicament delivery device when the actuator is moved to operate the dispensing mechanism such that the activation switch is urged to the on state and, preferably, said part comprises a part of the dispensing mechanism.

This configuration allows for actuation of the activation switch without requiring additional components. In some embodiments, said part comprises a drive sleeve of the dispensing mechanism.

In some embodiments, the activation switch is configured such that the activation switch moves a first distance from the off state to the on state and the sensor is configured such that the sensor moves a second distance from the idle position to the detecting position, wherein the second distance is greater than the first distance. This helps to ensure that the activation switch reaches the on position before the sensor reaches the detecting position.

In some embodiments, the dosage measurement system comprises first and second radial bearings, wherein the actuator is rotatably mounted to a part of the device via the first and second bearings, the first and second bearings being axially spaced. The part of the device may be, for example, a rotatable sleeve, for example, a dial sleeve, of the device or may be the housing.

In some embodiments, the dosage measurement system comprises a support member and a coupling member, wherein the dose setting dial is fixed relative to the support member and wherein the support member coupled to the coupling member and, preferably, the support member is coupled to the coupling member via a pivot.

In some embodiments, the support member is rotatably coupled to the coupling member via at least one radial bearing and, preferably, via first and second radial bearings. Optionally, the second radial bearing is axially spaced from the first radial bearing in the direction of actuation movement of the actuator.

In some embodiments, the dosage measurement system has any of the features discussed above. In some embodiments, there is provided a medicament delivery device comprising a dosage measurement system having any of the features discussed above.

According to another aspect, there is provided a dosage measurement system for a medicament delivery device, wherein the medicament delivery device comprises a reservoir for medicament and a dispensing mechanism operable to dispense medicament from the reservoir, the dispensing mechanism comprising a component configured to rotate during the dispensing of medicament, the component comprising a plurality of first and second encoder regions, the dosage measurement system comprising: first and second sensors that are offset such that, in use, rotation of the component causes one of the first encoder regions to align with the first sensor whilst one of the second encoder regions aligns with the second sensor and then one of the second encoder regions to align with the first sensor whilst one of the first encoder regions aligns with the second sensor, the first and second sensors configured to differentiate between the first and second encoder regions to detect rotation of the component; and, a processor configured to determine a dosage dispensed from the medicament reservoir based on the detected rotation of the component.

The provision of first and second offset sensors increases the resolution of measurement of the rotation of the component for a given size of encoding regions, allowing for a more accurate determination of the dosage dispensed from the medicament reservoir.

In some embodiments, the component comprises a plurality of formations, wherein each first encoding region comprises at least a portion of a respective formation that is detectable by the first and second sensors as the component rotates, wherein the second encoder regions are provided between adjacent first encoder regions.

In some embodiments, wherein each formation comprises a tooth, wherein each first encoding region comprises at least a portion of a respective tooth that is detectable by the first and second sensors as the component rotates, wherein each second encoder comprises at least a gap between adjacent teeth.

In some embodiments, the first and second sensors are arranged such that for all rotational positions of the component in which the first sensor aligns with one of the first encoding regions, the second sensor aligns with one of the second encoding regions.

This helps to ensure that the maximum resolution of measurement of rotation of the component can be achieved for a given number and size of encoding regions. Also, it is possible to ascertain that initial axial movement will cause a first encoding region to align with one of the first and second sensors and therefore false reading due to the initial axial movement can be disregarded.

In some embodiments, the component comprises a plurality of encoding periods, wherein each encoding period comprises one of the first encoding regions and an adjacent second encoding region, wherein for a given rotational position of the component the first sensor is aligned with a portion of one of the encoding periods and the second sensor is aligned with a different portion of one of the encoding periods.

In some embodiments, the dosage measurement system further comprises a third sensor that is aligned with a different portion of one of the encoding periods to the first and second sensors when the component is in said given rotational positon.

The processor is therefore able to determine the direction of rotation of the component.

In some embodiments, the dosage measurement system further comprises a fourth sensor that aligns with a different portion of one of the encoding periods to the first, second and third sensors when the component is in said given rotational positon. The processor is therefore able to determine the direction of rotation of the component.

In some embodiments, dosage measurement system further comprises a third sensor configured such that, in use, when the component is rotated the third sensor aligns with one of the first encoding regions at the same time that the first sensor aligns with one of the first encoding regions.

The third sensor provides redundancy should the first sensor fail to detect the encoding region due to a detecting error and also allows for improbable detection events to be filtered. Therefore, there is pairwise redundancy that allows for detection and compensation of erroneous switch state detection.

In some embodiments, the dosage measurement system further comprises a fourth sensor configured such that, in use, when the component is rotated the fourth sensor aligns with one of the first encoding regions at the same time that the second sensor aligns with one of the first encoding regions.

The fourth sensor provides redundancy should the second sensor fail to detect the encoding region due to a detecting error and also allows for improbable detection events to be filtered. Therefore, there is pairwise redundancy that allows for detection and compensation of erroneous switch state detection.

In some embodiments, when the first sensor is aligned with a transition between first and second encoding regions, the third sensor is also aligned with a transition between first and second encoding regions. In some embodiments, when the second sensor is aligned with a transition between first and second encoding regions, the fourth sensor is also aligned with a transition between first and second encoding regions.

In some embodiments, each of the first and second encoding regions extends about the rotational axis of the component by the same predetermined angle.

In some embodiments: said predetermined angle is about 15 degrees or 30 degrees.

In some embodiments, the second sensor is offset from the first sensor about the rotational axis in a first direction by an odd number integer multiple of the angle that each first encoding region subtends about the rotational axis.

In some embodiments, second sensor is offset from the first sensor about the rotational axis in the first direction by 165 degrees.

In some embodiments, each of the first and second encoding regions comprises a length that extends in the direction of rotation of the component, wherein the dosage measurement system comprises a third sensor that is offset from the first and second sensors such that, in use, when the first sensor is aligned with one of the first encoding regions and the second sensor is aligned with one of the second encoding regions, the third sensor is aligned with a different portion along the length of one of the first and second encoding regions.

In some embodiments, the processor is configured to determine a dosage dispensed from the medicament reservoir by a process including counting the number of transitions between the first and second encoding regions detected by the first and second sensors.

In some embodiments, the first and second sensors are configured to move from an idle position to a detecting position, wherein movement of the sensors to the detecting position causes one of the first and second regions to align with one of the first encoding regions, and wherein the processor is configured such that when determining the dosage dispensed the processor compensates for said alignment when the sensor moves to the detecting positon.

This allows for improved accuracy in embodiments wherein the sensors are moved axially into a detecting position. This is because, in some embodiments, such axial movement will cause one of the first and second sensors to align with a first encoding region which will appear as a false reading of a transition to said first encoding region. The processor can disregard this false reading to more accurately determine the dosage dispensed.

In some embodiments, the processor is configured to determine a dosage dispensed from the medicament reservoir based on a signal from one of the first and second sensors and an inverse signal from the other one of the first and second sensors.

In some embodiments, the processor is configured to determine a dosage dispensed from the medicament reservoir based on a superposition of the signal from one of the first and second sensors and the inverse signal from the other one of the first and second sensors.

In some embodiments, the processor is configured to determine a dosage dispensed from the medicament reservoir by comparing the superposition to a first threshold value and a second threshold value, greater than the first threshold value, and, preferably, counting the number of times the superposition transitions from a value lower than the first threshold value to a value greater than the second threshold value and/or from a value higher than the second threshold value to a value lower than the first threshold value.

This helps to improve the accuracy of the dosage determination by helping to filter out errors such as sensor noise and switch bouncing. Moreover, having four sensors may further improves accuracy because two simultaneous errors can be ignored.

In some embodiments, the dosage measurement system has any of the features discussed above.

In some embodiments, there is provided a medicament delivery system comprising the dosage measurement system.

These and other aspects of the disclosure will be apparent from and elucidated with reference to the embodiments described hereinafter.

### BRIEF DESCRIPTION OF THE FIGURES

Embodiments of the disclosure will now be described, by way of example only, with reference to the accompanying drawings, in which:

FIG. 1 is an exploded view of a medicament delivery device;

FIG. 2 is a cross-sectional side view of part of a medicament delivery device comprising a dosage measurement system according to an embodiment;

FIG. 3 is a cross-sectional side view of the medicament delivery device of FIG. 2, prior to the actuator being actuated by the user;

FIG. 4 is a cross-sectional side view of the device of FIG. 2, once the actuator has been actuated by the user;

FIG. 5 is a perspective view of an encoding ring of the device of FIG. 2;

FIG. 6 is a perspective view of a dial sleeve of the device of FIG. 2;

FIG. 7 is a perspective view of part of the dial sleeve and a sensor of the device of FIG. 2;

FIG. 8 is a side view of part of the dial sleeve and the sensor of the device of FIG. 2, with a sensing member in an unactuated state;

FIG. 9 is a perspective view of part of the dial sleeve and the sensor of the device of FIG. 2; with the sensing member in an actuated state;

FIG. 10 is a schematic side view of the sensor of the device of FIG. 2, showing the sensing member being actuated;

FIG. 11 is a schematic side view of first and second encoding regions of the dial sleeve of the device of FIG. 2;

FIG. 12 is a schematic side view of the first and second encoding regions actuating the sensor of the device of FIG. 2;

FIG. 13 is a cross-sectional side view of a portion of the device of FIG. 2, showing a portion of a support member and a coupling member;

FIG. 14 is a cross-sectional side view of a portion of the device of FIG. 2, showing a portion of the coupling member;

FIG. 15 is a schematic block diagram of the dosage measurement system of the device of FIG. 2;

FIG. 16A is a schematic view of sensors of another embodiment of dosage measurement system;

FIG. 16B is a schematic view of sensors of another embodiment of dosage measurement system;

FIG. 16C is a schematic view of sensors of another embodiment of dosage measurement system;

FIG. 17 shows signals output from the sensors of the embodiment of FIG. 16A;

FIG. 18 shows signals output from the sensors of the embodiment of FIG. 16A and a superposition signal;

FIG. 19 is a top view of the sensors and an activation switch of the embodiment of FIG. 16B;

FIG. 20 shows signals output from the sensors and activation switch of the embodiment of FIG. 16B and a superposition signal;

FIG. 21 is a top view of the sensors and an activation switch of the embodiment of FIG. 16C;

FIG. 22 is a perspective view of part of the dial sleeve and a sensor of another embodiment; and,

FIG. 23 is a circuit diagram of an embodiment of a dosage measurement system.

### DETAILED DESCRIPTION

In the following disclosure, embodiments will be described with reference to an insulin injection device. The present disclosure is however not limited to such application and may equally well be deployed with medicament delivery devices that eject other medicaments.

The term “distal” refers to a location that is relatively closer to a site of medicament delivery (for example, the injection site in the case of an injection device), and the term “proximal” refers to a location that is relatively further away from the site of medicament delivery.

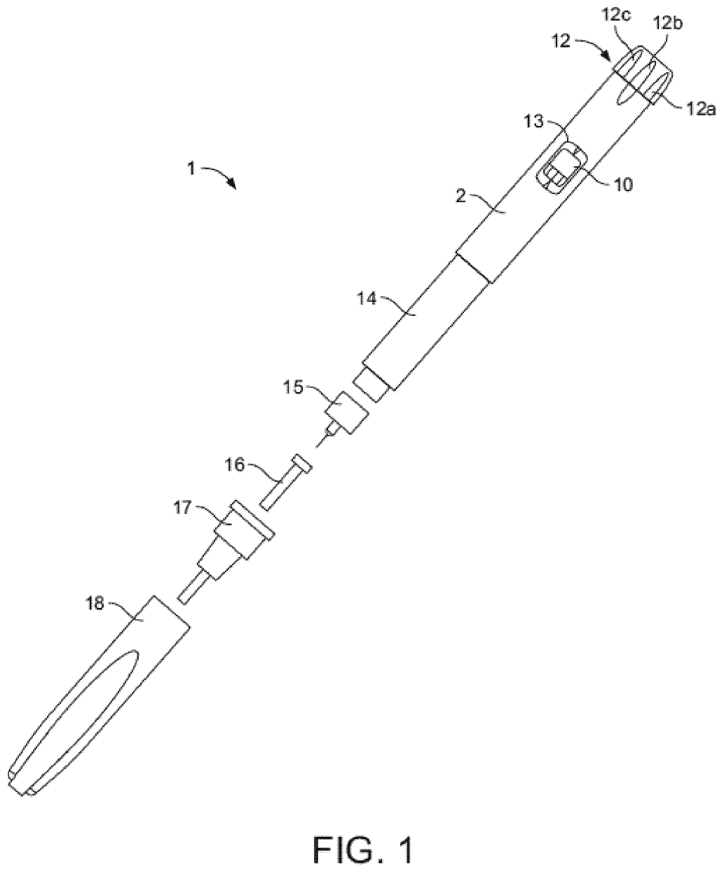

FIG. 1 is an exploded view of a medicament delivery device 1. In this example, the medicament delivery device 1 is an injection device 1, such as Sanofi's AllStar® insulin injection pen or Sanofi's SoloStar® insulin injection pen, however the present disclosure is also compatible with other types and makes of injection pens as described below. The present disclosure is also compatible with other types of medicament delivery devices such as needless injectors.

The injection device 1 of FIG. 1 is a pre-filled injection pen that comprises a housing 2 and contains a reservoir 14 for medicament which, in the present embodiment, is an insulin container 14. A needle 15 can be affixed to the reservoir 14. The injection device 1 may be disposable or re-usable. The needle 15 is protected by an inner needle cap 16 and an outer needle cap 17 and/or an alternative cap 18.

An insulin dose to be ejected from injection device 1 can be programmed by moving a dose setting member 12, which in the present embodiment is a dose setting dial 12 that can be turned relative to the housing 2 to allow for the dose to be ‘dialed in’. A currently programmed dose is then displayed via dosage window 13, for instance in multiples of units. For example, where the injection device 1 is configured to administer human insulin, the dosage may be displayed in so-called International Units (IU), wherein one IU is the biological equivalent of about 45.5 micrograms of pure crystalline insulin ( 1/22 mg). Other units may be employed in medicament delivery devices for delivering analogue insulin or other medicaments.

The dosage window 13 may be in the form of an aperture in the housing 2, which permits a user to view a limited portion of a number sleeve 10 that is configured to move when the dose setting dial 12 is turned, to provide a visual indication of a currently programmed dose. It should be noted that the selected dose may equally well be displayed differently than as shown in the dosage window 13 in FIG. 1. For example, only every second dose unit may be shown, due to space restrictions on the number sleeve 10. The un-numbered dose units may be represented by tick marks between the displayed numerals. Alternatively, the number sleeve 10 may remain stationary during the dose dialling phase, and the dosage window 13 may move as a dose is dialed in to reveal the number corresponding to the dialed dose. In either case, the number sleeve 10 may be a component which rotates when a dose is being dispensed from the injection device 1.

In this example, the dose setting dial 12 includes one or more formations 12a, 12b, 12c that facilitate programming because they improve the grip a user feels when grasping the dose setting dial 12. In another example (not shown) the dose setting dial does not include formations.

The injection device 1 may be configured so that turning the dose setting dial 12 causes a mechanical click sound to provide acoustical feedback to a user. The number sleeve 10 mechanically interacts with a piston in insulin container 14. When needle 15 is stuck into a skin portion of a patient, and then the dose setting dial 12 is pushed axially relative to the housing 2, the insulin dose displayed in display window 13 will be ejected from injection device 1. The dose setting dial 12 therefore forms an actuator that may be actuated by the user to dispense medicament. When the needle 15 of injection device 1 remains for a certain time in the skin portion after the dose setting dial 12 is pushed, a high percentage of the dose is actually injected into the patient's body. Ejection of the insulin dose may also cause a mechanical click sound, which is however different from the sounds produced when rotating the dose setting dial 12 to set the dose to be delivered.

The injection device 1 comprises a dial sleeve 10 which, in the present embodiment, is the same component as the number sleeve 10. In other embodiments (not shown), the dial sleeve 10 may be secured to the proximal end of the number sleeve 10.

The dial sleeve 10 has a proximal end close to the dose setting dial 12. The dial sleeve 10 (and the number sleeve 10, which is the same component) rotates relative to the dose setting dial 12 during dose ejection, but not during dose dialling. During dose dialling, the user rotates the dose setting dial 12 relative to the housing 2, which causes corresponding rotation of the dial sleeve 10 relative to the housing 2.

In order to operate the injection device 1 to dispense medicament from the container 14, the dose setting dial 12 is configured to move axially relative to the housing 2 and dial sleeve 10 of the injection device 1 by a short distance. This movement occurs when a user exerts a force on the end of the dose setting dial 12. For example, the user pushing the does setting dial 12 axially towards the injection site. This movement disengages a clutch (not shown) and allows the dial sleeve 10 and other internal components of the injection device 1 to rotate relative to the dose setting dial 12. Alternatively, the injection device 1 may comprise a separate injection button (not shown in FIG. 1) that is mounted to the dose setting dial 12 and which moves axially relative to the housing 2 in order to cause the medicament to be dispensed. The injection button may be located at the proximal end of the dose setting dial 12.

In the various embodiment, during delivery of the insulin dose the dose setting dial 12 is moved to its initial position in an axial movement, that is to say without rotation, while the number sleeve 10 is rotated to return to its initial position, e.g. to display a dose of zero units.

The injection device 1 may be used for several injection processes until either the insulin container 14 is empty or the expiration date of the medicament in the injection device 1 (for example, 28 days after the first use) is reached.

Before using injection device 1 for the first time, it may be necessary to perform a so-called “prime shot” to remove air from insulin container 14 and needle 15, for instance by selecting two units of insulin and pressing the dose setting dial 12 while holding the injection device 1 with the needle 15 pointing upwards. For simplicity of presentation, in the following, it will be assumed that the ejected doses substantially correspond to the injected doses, so that, for instance the number of medicament dose units ejected from the injection device 1 is equal to the number of medicament dose units received by the user. Nevertheless, differences (e.g. losses) between the ejected amounts and the injected doses may need to be taken into account in some applications of the device.

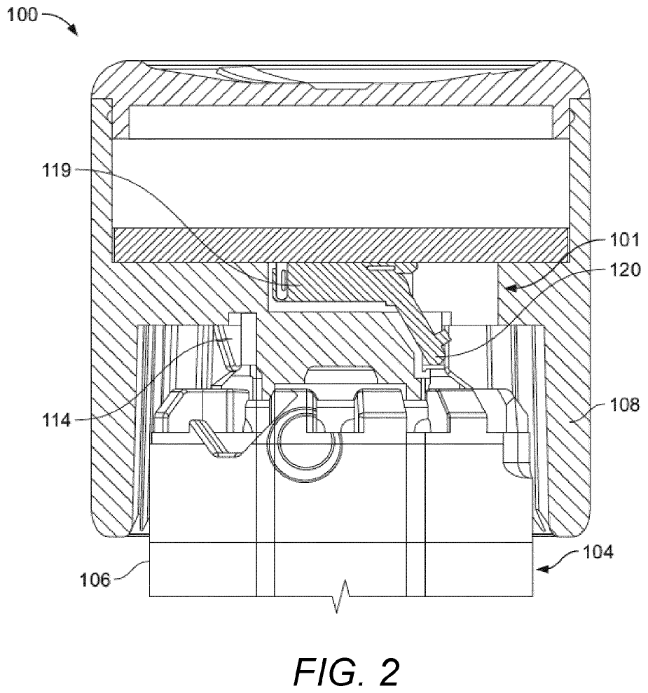

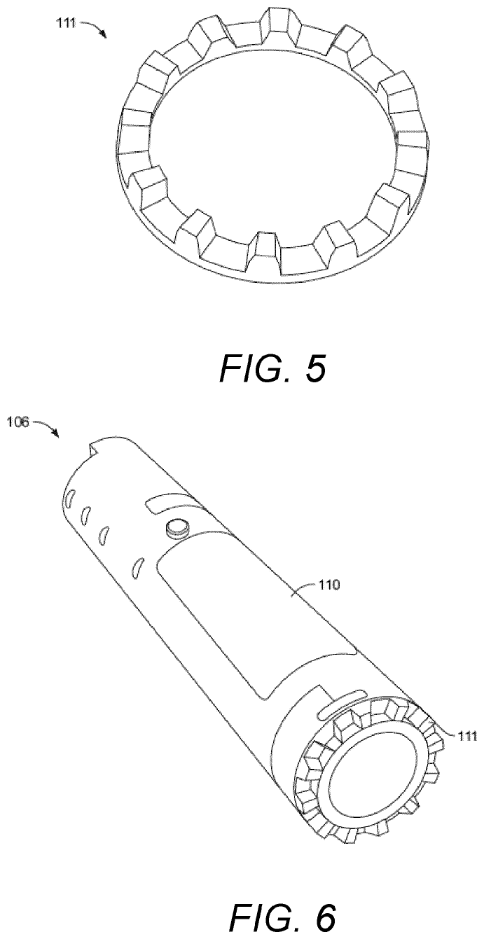

Referring now to FIGS. 2 to 15, an embodiment of a medicament delivery device 100 is shown. The medicament delivery device 100 is similar to the medicament delivery device 1 described in relation to FIG. 1, and thus a detailed description of common features of the medicament delivery device 100 will not be repeated hereinafter. A difference is that the medicament delivery device 100 comprises a dosage measurement system 101.

The medicament delivery device 100 comprises a housing 102 containing a reservoir (not shown) for medicament. The medicament delivery device 100 further comprises a dispensing mechanism 104 operable to dispense medicament from the reservoir.

The dispensing mechanism 104 comprises a clutch 105, dial sleeve 106, a drive sleeve 107, a drive member (not shown), a plunger rod (not shown) and a piston (not shown). The drive member may comprise a biasing member (not shown), for example, a spring, and is configured to bias the drive sleeve 107 to rotate relative to the housing 102.

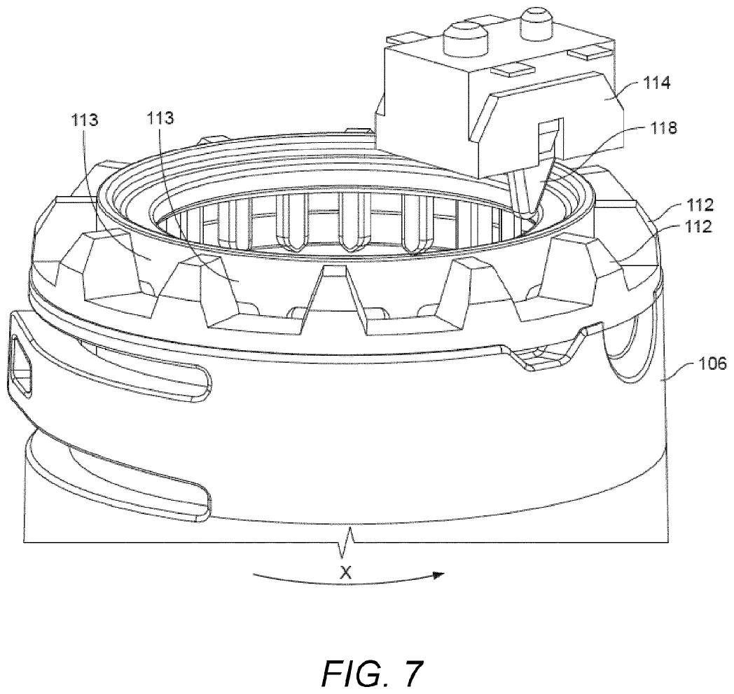

The drive sleeve 107 is coupled to the plunger rod such that rotation of the drive sleeve 107 in a first rotational direction (shown by arrow ‘X’ in FIGS. 7 to 11) causes the plunger rod to move axially in the distal direction to dispense medicament from the reservoir. More specifically, the piston (not shown) is mounted to the distal end of the plunger rod such that axial movement of the plunger rod in the distal direction during rotation of the drive sleeve 107 in the first rotational direction X causes the piston to slide distally within the reservoir to dispense medicament therefrom.

In one embodiment, the drive sleeve 107 and plunger rod comprise corresponding threads (not shown) that engage so that rotation of the drive sleeve 107 causes axial movement of the plunge rod. In other embodiments (not shown), the drive sleeve 107 is coupled to the plunger rod via one or more intermediate components (not shown).

The clutch 105 is initially in an engaged position in which the clutch 105 prevents the drive sleeve 107 from rotating in the first rotational direction X under the force of the drive member. In some embodiments, the clutch 105 has one or more splines (not shown) or other engaging elements that engage with the drive sleeve 107 or another component when the clutch 105 is in the engaged position to prevent rotation of the drive sleeve 107 relative to the housing 102.

The medicament delivery device 100 further comprises a dose setting member 108, which in the present embodiments is a dose setting dial 108 that is mounted to the proximal end of the housing 102. An medicament dose to be expelled from medicament delivery device 100 can be programmed, or ‘dialed in’ by turning a dose setting dial 108, and a currently programmed dose is then displayed via dosage window (not shown), for instance in multiples of units. In some embodiments, the dose setting dial 108 is rotated in a second rotational direction, opposite to the first rotational direction X, relative to the housing 102 such that the dial sleeve 106 is also rotated in the second rotational direction relative to the housing 102 from an initial position (representing a dose of ‘zero’) until the desired dose is selected.

The dial sleeve 106 has a proximal end close to the dose setting dial 108. The dial sleeve 106 (and the number sleeve 106, which is the same component) rotates relative to the dose setting dial 108 during dose ejection, but not during dose dialling. In other embodiments (for example, embodiments wherein the dose setting dial 108 comprises a separate actuator such as a push button, the dial sleeve 106 does not rotate relative to the dose setting dial 108 during dose ejection and instead the dose setting dial 108 rotates together with the dose sleeve 106, optionally both the dose setting dial 108 and dial sleeve 106 rotating relative to the actuator).

During dose dialling, the user rotates the dose setting dial 108 relative to the housing 102 in the second rotational direction, which causes corresponding rotation of the dial sleeve 106 relative to the housing 102.

The dispensing mechanism 104 further comprises a number sleeve 106 which in the present embodiment is the same component as the dial sleeve 106. In other embodiments (not shown), the dial sleeve may be secured to the number sleeve.

The user will be able to determine that the desired dose has been selected by viewing the number sleeve 106 through the viewing window (not shown) in the housing 102.

The dose setting dial 108 forms an actuator that may be actuated by the user to dispense medicament. In more detail, the dose setting dial 108 is slidable axially relative to the housing 102 to operate the dispensing mechanism 104 to dispense medicament from the reservoir. In some embodiments, rotation of the dose setting dial 108 in the second rotational direction during dose dialling causes the dose setting dial to move axially in the proximal direction. In other embodiments, rotation of the dose setting dial 108 in the second rotational direction during dose dialling does not cause the dose setting dial 108 to move axially.

In operation the dose setting dial 108 is configured to move axially relative to the housing 102 by a short distance. This movement occurs when a user exerts a force on the dose setting dial 108, for example, the user pushing the dose setting dial 108 axially in the distal direction, towards the injection site. This axial movement of the dose setting dial 108 disengages the clutch 105 such that the clutch 105 moves to a disengaged position. For example, said distal movement of the dose setting dial 108 may cause a corresponding distal movement of the clutch 105 that disengages the clutch 105, for instance, disengaging the splines or other engaging elements (not shown). Disengaging the clutch 105 allows the dial sleeve 106 and drive sleeve 107 to rotate relative to the housing 102 and dose setting dial 108 in the first rotational direction X under the force of the drive member to dispense medicament from the reservoir.

As long as the dose setting dial 108 is kept depressed by the user, the clutch 105 will remain in the disengaged positon and thus the dial sleeve 106 and drive sleeve 107 will continue to rotate in the first rotational direction X under the force of the drive member to deliver medicament to the user until the dial sleeve 106 reaches the initial position. The dose setting dial 108 may be biased proximally by a dial biasing member (not shown).

The dial sleeve 106 comprises a cylindrical body 110 and an encoder ring 111 that is provided on the proximal end of the body 110. In the present embodiment, the encoder ring 111 is attached to the body 110, but in other embodiments the encoder ring 111 may be integrally formed with the body 110 (for instance, the encoder ring 111 and body 110 being moulded or cast from a single portion of material).

The encoding ring 111 comprises a plurality of formations 112 which, in the present embodiment, are teeth 112 with gaps therebetween 113. The teeth 112 extend proximally.

The dosage measurement system 101 further comprises a mechanically actuated sensor 114 configured such that, in use, rotation of the dial sleeve 106 in the first rotational direction X causes successive formations 112 to engage the sensor 114 such that the sensor 114 detects rotation of the dial sleeve 106.

The dosage measurement system 101 further comprises one or more processors 115 configured to determine a dosage dispensed from the medicament reservoir based on the detected rotation of the dial sleeve 106. The processors 115 may include, for example, a microprocessor, a Digital Signal Processor (DSP), Application Specific Integrated Circuit (ASIC), Field Programmable Gate Array (FPGA) or the like.

The dosage measurement system 101 further comprises one or more computer readable memory media. In the present embodiment, the computer readable memory media comprises memory units 116A, 116B, including program memory 116A and main memory 116B, which can store software for execution by the processor 115.

The dosage measurement system 101 further comprises a battery 109 that is configured to power the dosage measurement system 101.

The dosage measurement system 100 further comprises an output 117. The output 117 may be a wireless communications interface for communicating with another device via a wireless network such as wi-fi or Bluetooth®, or an interface for a wired communications link, such as a socket for receiving a Universal Series Bus (USB), mini-USB or micro-USB connector.

In the present embodiment, the sensor 114 is a mechanically actuated switch 114. In one particular embodiment, the sensor 114 is a C&K (TM) HDT0001 switch.

In some embodiments, the switch 114 is moved from one of an on or off state to the other one of an on or off state when a formation 112 engages with the switch 114 during rotation of the dial sleeve 106.

The sensor 114 comprises a sensing member 118 that is configured to move from an unactuated state to a actuated state when the sensing member 118 is engaged by one of the formations 112 during rotation of the dial sleeve 106. The sensor 114 is configured to detect movement of the sensing member 118 between the unactuated and actuated states. The sensing member 118 may be a pivotal member 118 that rotates when the sensing member 118 is engaged by one of the formations 112 during rotation of the dial sleeve 106.

In some embodiments, each formation 112 comprises a leading edge 112A arranged such that when the dial sleeve 106 rotates in the first rotational direction X the leading edge 112A of each formation 112 abuts the sensor 114 in turn. In some embodiments, engagement of the sensor 114 with the leading edge 112A of a first of the formations 112 until engagement of the sensor 114 with the leading edge 112A of an adjacent second of the formations 112 during rotation of the dial sleeve 106 represents one encoding period.

In some embodiments, the sensor 114 is actuated for between 40 to 60% of the encoding period. Preferably, the sensor 114 is actuated for about 50% of the encoding period. When the dial sleeve 106 is rotated in the first rotational direction X during the dispensing of medicament, for 50% of the angular positions of the dial sleeve 106 in one full rotation the sensor 114 is actuated and for the remaining 50% of the angular positions of the dial sleeve 106 the sensor 114 is not actuated.

In some embodiments, the medicament delivery device 100 comprises a torque limiter (not shown). The sensor 114 is mounted to the dose setting dial 108 via the torque limiter such that rotation of the dose dial 108 relative to the housing 102 with a torque greater than a predetermined limit causes the torque limiter to move to an open state such that the dose setting dial 108 rotates relative to the sensor 114. Optionally, the torque limiter comprises an overload clutch (not shown).

As explained above, in some embodiments pressing the dose setting dial 108 axially relative to the housing 102 disengages the clutch 105 to allow the dial sleeve 106 and drive sleeve 107 to rotate relative to the housing 102 and dose setting dial 108. In some embodiments, with the dose setting dial 108 depressed and the clutch 105 in a disengaged state it may be possible for the user to grip the dial sleeve 106 and rotate it in the second rotational direction relative to the dose setting dial 108, against the force of the drive mechanism. This unintended operation of the device 100 could cause damage to the device 100. In particular, the sensing member 118 of the sensor 114 may be shaped and arranged to detect rotation of the dial sleeve 106 in the first rotational direction relative to the dose setting dial 108, that is, such that the sensing member 118 moves over the formations 112 without snagging. If instead the dial sleeve 106 is forced in the second rotational direction relative to the dose setting dial 108 then this may cause the formations 112 of the dial sleeve 106 to snag on the sensing member 118 of the sensor 114, which could cause damage to the sensing member 118 or misalignment of the sensing member 118 relative to the formations 112.

In some embodiments, the medicament delivery device 100 comprises a one-way mechanism (not shown). The one-way mechanism may be configured to alleviate the above situation.

In one embodiment, the sensor 114 is mounted to the dose setting dial 108 via the one-way mechanism such that rotation of the sensor 114 relative to the dose setting dial 108 is resisted by the one-way mechanism. Thus, under normal operational conditions when the clutch 105 is disengaged and the dial sleeve 106 rotates in the first rotational direction X during the dispensing of medicament, the formations 112 move over the sensing member 114 and the sensing member 114 is held stationary by the one-way mechanism such that the relative rotation of the formations 112 and sensing member 114 can be detected.

The one-way mechanism is configured to permit rotation of the sensor 114 relative to the dose setting dial 108 in the second rotational direction. Therefore, in the unintended operational scenario discussed above wherein the user disengages the clutch 105 and then forces the dial sleeve 106 in the second rotational direction, the formations 112 will be urged against the sensing member 118 and will urge the sensor 114 to also rotate in the second rotational direction. The one-way mechanism permits rotation of the sensor 114 in the second rotational direction and therefore the sensor 114 will rotate together with the dial sleeve 106 in the second rotational direction relative to the dose setting dial 108 and therefore the formations 112 will not be forced over the sensing member 118 and thus damage to the sensing member 118 and/or formations 112 will be prevented. In some embodiments, the one-way mechanism may comprise ratchet teeth on one or both of the dose setting dial 108 and sensor 114.

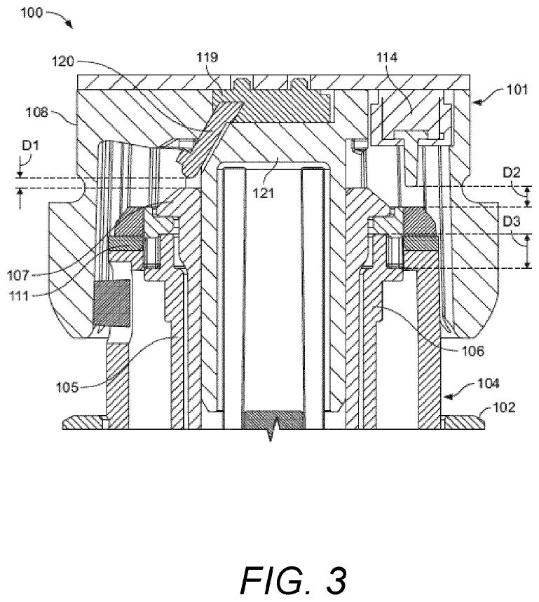

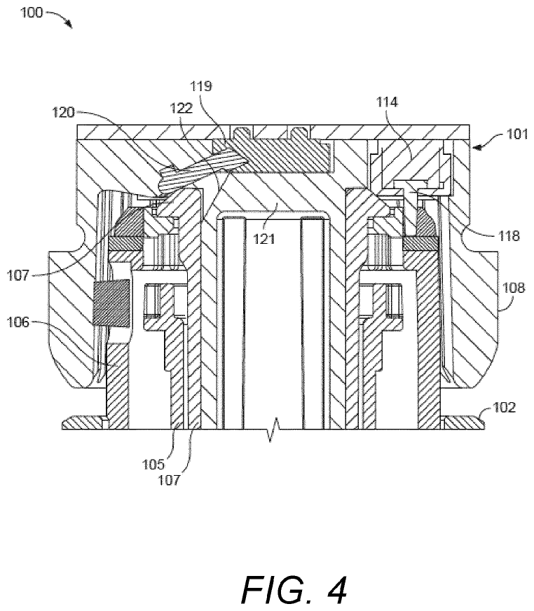

The sensor 114 is mounted to the dose setting dial 108 such that axial movement of the dose setting dial 108 relative to the housing 102 to operate the dispensing mechanism 104 to dispense medicament also moves the sensor 114 axially. More specifically, the sensor 114 moves from an idle position (shown in FIG. 3), wherein the sensor 114 is spaced from the formations 112 in the axial direction of the medicament delivery device 100, to a detecting position (shown in FIG. 4), wherein the sensor 114 is moved distally such that rotation of the dial sleeve 106 causes successive formations 112 to be detected by the sensor 114 such that the sensor 114 detects rotation of the dial sleeve 106. In the present embodiment in which the sensor 114 comprises a mechanical switch 114, when the sensor 114 is in the detecting position the formations 112 physically engage with the sensor 114 as the dial sleeve 106 rotates in the first rotational direction X.

The medicament delivery device 100 further comprises an activation switch 119 that is moveable from an initial off state (shown in FIG. 3) to an on state to activate the dosage measurement system 101 upon actuation of the dose setting dial 108. For example, movement of the activation switch 119 to the on state may wake-up or power-on one or more of the sensor 114, the processor 115 or other components of the dosage measurement system 101.

In the present embodiment, the activation switch 119 is mounted to the dose setting dial 108 such that the activation switch 119 moves together with the dose setting dial 108 such that the activation switch 119 is urged against a component of the medicament delivery device 100, for example, the drive sleeve 107, to move the activation switch 119 to the on state.

In another embodiment (not shown), the activation switch 119 is mounted to a component of the medicament device 100 other than the dose setting dial 108, wherein sliding movement of the dose setting dial 108 during actuation causes the dose setting dial 108, or a part connected to the dose setting dial 108, to be urged against the activation switch 119 to move the activation switch 119 to the on state. For instance, the activation switch 119 may be mounted to the proximal end of the drive sleeve 107.

The sensor 114 and activation switch 119 are configured such that, when the dose setting dial 108 is actuated by the user sliding the dose setting dial 108 in the distal direction, the activation switch 119 reaches the on state prior to the sensor 114 reaching the detecting position. This helps to ensure that the dosage measurement system 101 has time to power-on before the sensor reaches 114 the detecting position to detect rotation of the dial sleeve 106. Thus, the accuracy of the measurement of the dose dispensed by the dispensing mechanism 104 is increased.

The skilled person will recognise that many different types of activation switch 119 may be used, for example, a relay, analogue switch, pressure sensor, push switch or flick switch. In one embodiment, the activation switch 119 comprises a Panasonic™ ESE13 switch.

In the present embodiment, the activation switch 119 comprises a pivotal member 120 that pivots from the off state to the on state to activate the dosage measurement system 101. The pivotal member 120 is pivotally connected to the dose setting dial 108.

The activation switch 119 is configured to engage a part of the medicament delivery device 100 when the dose setting dial 108 is moved axially to operate the dispensing mechanism 104 such that the activation switch 119 is urged to the on state. In some embodiments, said part comprises a part of the dispensing mechanism 104, for example, the drive sleeve 107. In some embodiments in which the activation switch 119 comprises a pivotal member 120, the pivotal member 120 engages with said part of the medicament delivery device 100 when the dose setting dial 108 is moved axially such that the pivotal member 120 rotates relative to the dose setting dial 108 from the off state to the on state.

In some embodiments, the medicament delivery device 100 comprises a stop 122 and wherein the activation switch 119 is configured to rest against the stop 122 when the activation switch 119 is in the off state.

In some embodiments, the medicament delivery device 100 comprises a support member 121. The support member 121 projects axially from the dose setting dial 108 in the distal direction. The support member 121 is generally cylindrical and is configured to be received within a bore of the drive sleeve 107. The support member 121 moves into the drive sleeve 107 when the dose setting dial 108 is actuated. The support member 121 may be attached to or integrally formed with the dose setting dial 108. In one embodiment, the support member 121 is mounted to the activation switch 119.

Optionally, the support member 121 comprises a stop 122 against which the activation switch 119 abuts when the activation switch 119 is in the off state. The stop 122 may be in the form of a stop surface 122 of the support member 121. In the present embodiment, the pivotal member 120 abuts the stop surface 122 when the activation switch is in the off state. The pivotal member 120 rotates away from the stop surface 122 when the pivotal member 120 is urged against the drive sleeve 107 during actuation of the actuator 109. Once the activation switch 119 reaches the on state, the pivotal member 120 can further rotate to accommodate additional axial movement of the dose setting dial 108 relative to the housing 102 in the distal direction so that the dosage measurement system 101 remains powered-on. In other embodiments, the activation switch 119 only needs to move to the on state briefly to power-on the dosage measurement system 101, after which point the dosage measurement system 101 will remain powered-on until the battery is depleted or for a set period of time regardless of the subsequent position of the activation switch 119.

In some embodiments, the activation switch 119 is configured such that the activation switch 119 moves a first distance D1 (as illustrated in FIG. 3) from the off state to the on state and the sensor 114 is configured such that the sensor 114 moves a second distance D2 (as illustrated in FIG. 3) from the idle position to the detecting position. The second distance D2 is greater than the first distance D1 such that the activation switch 119 reaches the on state before the sensor 114 reaches the detecting position. The dose setting dial 108 must move axially the first distance D1 to move the activation switch 119 to the on state and must move axially the second distance D2 to move the sensor 114 to the detecting positon. The first and second distances D1, D2 may both extend in the axial direction of the medicament delivery device 100.

In FIG. 3, the activation switch 119 moves from the off state to the on state to power the processor as soon as the pivotal member 120 is urged against the dial sleeve 107. However, it should be recognised that in alternative embodiments (not shown), the pivotal member 120 rotates partially, or fully, after abutment with the dial sleeve 107 before the activation switch moves 119 to the on state and the processor is powered on. The point at which the activation switch 119 moves to the on state is the switch point of the activation switch 119.

Similarly, in FIG. 3, the sensor 114 moves from the idle position to the detecting position as the sensing member 118 overlaps one of the formations 112 in the axial direction of the device 100 such that, if the sensor 114 is rotationally aligned with a formation 112, the sensing member 118 will begin to rotate and this is detected by the processor. However, it should be recognised that in alternative embodiments (not shown), the sensing member 118 rotates partially, or fully, after abutment with a formation 112 before this movement is detected, and this position of the sensing member 118 is the switch point of the sensing member 118. In other words, the detecting position of the sensor 114 may be the position wherein the switch point of the sensing member 118 is axially aligned with the formations 112.

Upon movement of the dose setting dial 108 axially, the switch point of the activation switch 119 is reached before the switch point of the sensor 114.

In some embodiments, the dose setting dial 108 must move axially a third distance D3 (as illustrated in FIG. 3) to operate the dispensing mechanism 104 to dispense medicament, for instance, to disengage the clutch 105. The third distance D3 may be larger than the first and second distances D1, D2 to help ensure that the medicament is not dispensed from the reservoir until after the activation switch 119 has been moved to the on state and the sensor 114 moved to the detecting position.

In some embodiments, the support member 121 is rotatably coupled to a component of the medicament delivery device 100 via first and second bearings 125, 126. The first and second bearings 125, 126 may be radial bearings. However, it should be recognised that one of the first and second radial bearings 125, 126 may be omitted.

In the present embodiment, the support member 121 is rotatably coupled to a coupling member 127 via the first and second bearings 125, 126. However, it should be recognised that in other embodiments the coupling member 127 is omitted.

The first bearing 125 comprises a curved inner surface 121A of the support member 121 and a curved outer surface 127A of the coupling member 127. The inner and outer surfaces 121A, 127A may be cylindrical. The inner and outer surfaces 121A, 127A may extend circumferentially about the longitudinal axis of the medicament delivery device 100.

The inner and outer surfaces 121A, 127A engage to form the first radial bearing 125 such that the support member 121 can rotate relative to the coupling member 127, with the inner surface 121A of the support member 121 gliding over the outer surface 127A of the coupling member 127.

The second bearing 126 comprises a circumferentially extending groove 121B of the support member 121 and a rib 127B of the coupling member 127. The rib 127B is received within the groove 121B. The rib 127B and groove 121B form the second radial bearing 126 such that the support member 121 can rotate relative to the coupling member 127, with the rib 127B rotating within the groove 121B. In some embodiments, the engagement of the rib 127B and groove 121B retains the support member 121 and coupling member 127 axially relative to each other.

It should be recognised that in alternative embodiment (not shown), the support member 121 may comprise a groove that receives a rib of the coupling member 127 to form the second bearing 126.

The coupling member 127 is rotationally fixed relative to the dial sleeve 106. The coupling member 127 may be slidably mounted to the sleeve 106 such that the coupling member 127 is axially moveable together with the dose setting dial 108 during actuation. For example, the coupling member 127 may be slidably mounted on a longitudinal member that extends distally from the coupling member 127 and is connected to an internal part of the medicament delivery device 100. In another embodiment, the coupling member 127 is mounted to the clutch 105 such that actuation of the dose setting dial 108 urges the coupling member 127 axially, which in turn urges the clutch 105 axially to disengage.

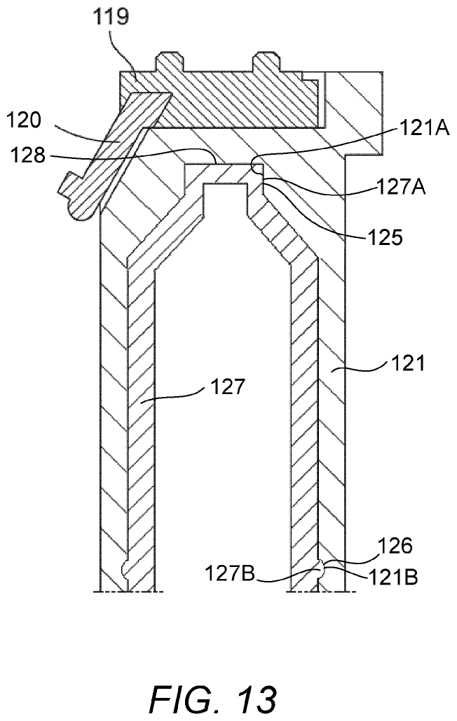

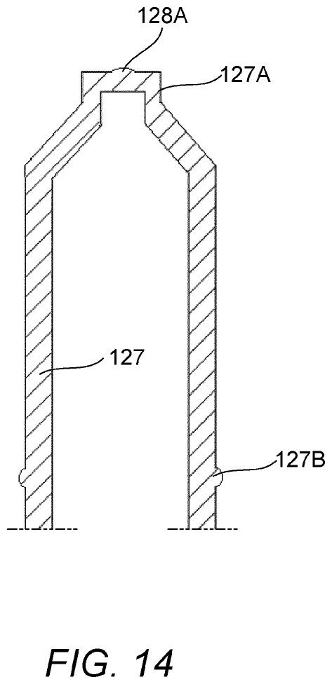

Optionally, the device 100 further comprises a pivot 128. In FIG. 14, the pivot 128 is shown in the form of a pivot point 128A comprising a rounded protrusion 128A that extends proximally from the proximal end of the coupling member 127 (the pivot point 128A is not shown in FIG. 13). The protrusion 128A abuts a generally flat distally facing surface of the support member 121. In alternative embodiment (not shown), the rounded protrusion is provided on the distally facing surface of the support member 121 and abuts a proximally facing surface of the coupling member 127.

The pivot 128 is configured to allow for rotation of the dose setting dial 108 relative to the coupling member 127, for example, due to rotation of the coupling member 127 relative to the dose setting dial 108 during the dispensing of medicament. The pivot 128 helps to minimise the surface area of contact between the support member 121 and the coupling member 127 and thus to reduce friction therebetween.

The pivot 128 is configured such that if the user exerts a force on the dose setting dial 108 that urges the dose setting dial 108 to tilt relative to the housing 102, for example, by applying an off-centre force at a peripheral edge of the dose setting dial 108, then the dose setting dial 108 will be urged to tilt about the pivot 128. However, the first and second radial bearings 125, 126 are configured to resist said tilting of the dose setting dial 108 and thus the radial bearings 125, 126 help to maintain the dose setting dial 108 in alignment with the housing 102.

In more detail, it is desirable to prevent tilting of the dose setting dial 108 relative to the housing 102, which could otherwise cause misalignment of the sensor 114 relative to the formations 112. For instance, if the user presses on an edge of the dose setting dial 108 during actuation, this could cause the dose setting dial 108 to be urged to tilt relative to the longitudinal axis of the medicament delivery device 100, such that the proximal end of the dose setting dial 108 is no longer perpendicular to the longitudinal axis and is now instead at an angle thereto. To help prevent this, the second bearing 126 is located distally of the first bearing 125. Therefore, if the user exerts a force on the dose setting dial 108 that causes the dose setting dial 108 to tilt, the second bearing 126 will provide a reaction force that prevents tilting of the dose setting dial 108. The second bearing 126 being spaced from the pivot 128 means that the reaction force needed to be exerted by the second bearing 126 to stabilise the dose setting dial 108 and prevent tilting is smaller than if the second bearing 126 was located closer to the pivot 128.

The second bearing 126 may be located radially outwards of the first bearing 125, in other words, further away from the longitudinal axis of the medicament delivery device 100 than the first bearing 125. That is, the second bearing 126 may have a larger diameter than the first bearing 125. Thus, the reaction torque exerted by the first bearing 125 if the dose setting dial 108 is rotating whilst tilted is reduced in comparison to if the first bearing 125 had a larger diameter. This reduces friction resisting rotation of the dose setting dial 108. The second bearing 126 has a larger diameter than the first bearing 125 to provide support when the dose dial setting 108 is tilted about the pivot 128.

Optionally, the support member 121, or another component, may comprise the aforementioned stop 122 against which the activation switch 119 abuts when in the off state.

Operation of the medicament delivery device 100 will now be described. The user dials in a dose to be delivered by rotating the dose setting dial 108 relative to the housing 102 in the second rotational direction, which causes corresponding rotation of the dial sleeve 106 relative to the housing 102 in the second rotational direction until the desired dose is displayed in the dosage window (not shown). The needle (not shown) is inserted into the injection site of the patient.

To deliver the dose of medicament, the user pushes the dose setting dial 108 axially into the housing 102 in the distal direction. This causes the sensor 114 and activation switch 119 mounted to the dose setting dial 108 to move axially in the distal direction. The axial movement of the dose setting dial 108 first causes the activation switch 119 to abut the drive sleeve 107 such that the activation switch 119 is moved to the on state and thus the processor 115 of the dosage measurement system 101 is powered-on.

Continued movement of the dose setting dial 108 in the distal direction causes the sensor 114 to move to the detecting position, wherein the sensor 114 overlaps the formations 112 in the axial direction of the medicament delivery device 100, and then the dispensing mechanism 104 is operated to expel medicament from the reservoir for delivery via the needle. For instance, the medicament delivery device 100 may comprise a release mechanism that is activated to release the drive member when the dose setting dial 108 has been moved distally to an axial position in which the sensor 114 is in the detecting position, or in other embodiments the dose setting dial 108 has been moved further distally in the axial positon (e.g. by a third distance D3).

In the present embodiment, movement of the dose setting dial 108 by the third distance D3 relative to the housing 102 causes the clutch 105 to be disengaged such that the clutch 105 moves to a disengaged position. Thus, the dial sleeve 106 and drive sleeve 107 are allowed to rotate relative to the housing 102 and dose setting dial 108 in the first rotational direction X under the force of the drive member to dispense medicament from the reservoir. In another embodiment (not shown), the clutch 105 is disengaged when the dose setting dial 108 has been moved by the second distance D2 relative to the housing 102 at the same time that the senor 114 reaches the detecting position.

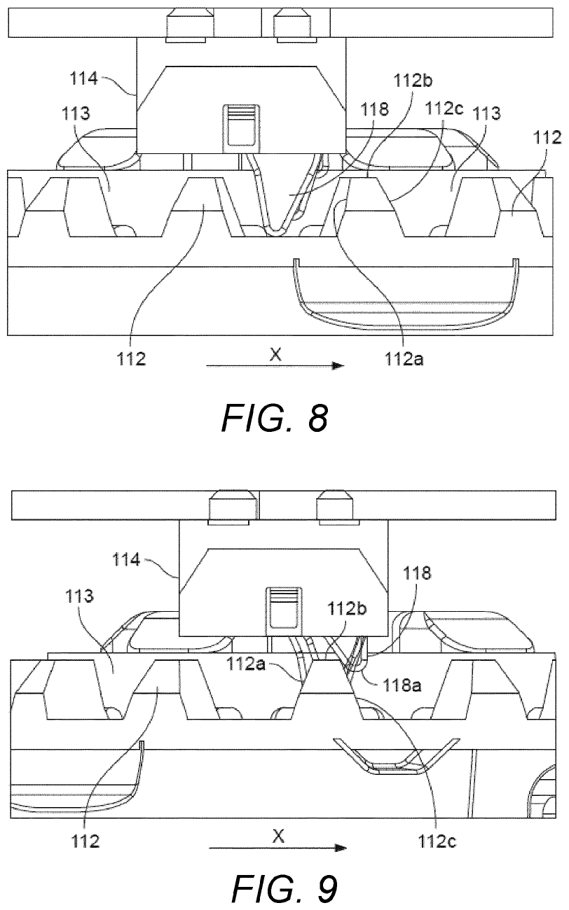

The rotation of the dial sleeve 106 in the first rotational direction X relative to the housing 102 during the dispensing of medicament is detected by the sensor 114 such that the processor 115 can determine the dosage delivered by the medicament delivery device 100. In particular, rotation of the dial sleeve 106 causes successive formations 112 to be urged against the sensing member 118 such that the sensing member 118 is repeatedly moved between the unactuated state (shown in FIG. 8) and the actuated state (shown in FIG. 9).

The processor 115 is configured to determine the delivered dose based on the movement of the sensing member 118. For example, the processor 115 may be configured to count the number of times that the sensing member 118 transitions from the unactuated state to the actuated state and/or the number of times that the sensing member 118 transitions from the actuated state to the unactuated state. The memory 116A, 116B may be programmed with information or instructions allowing the processor 115 to convert the number of transitions into a dosage delivered.

As the dial sleeve 106 rotates in the first rotational direction X, a portion of the leading edge 112A of a first formation 112 will abut the sensing member 118 and urge the sensing member 118 from the unactuated state to the actuated state such that the sensor 114 transitions from outputting a LOW signal to outputting a HIGH signal. This represents the start of a first encoding region 123 and the end of a second encoding region 124. The formation 112 will remain engaged with the sensing member 118 as the dial sleeve 106 continues to rotate in the first rotational direction X such that the sensing member 118 remains in the actuated state. As the formation 112 starts to pass the sensor 114, the sensing member 118 will begin to move back towards the unactuated state whilst still being engaged with the formation 112. In some embodiments, the sensing member 118 is biased into the unactuated state by a biasing member (not shown), for example, a spring.

With further rotation of the dial sleeve 106 in the first rotational direction X, the sensing member 118 will reach the unactuated state and thus the sensor 114 will transition from outputting a HIGH signal to outputting a LOW signal. This represents the end of the first encoding region 123 and the start of a second encoding region 124. The sensor 114 will then be aligned a gap 113 adjacent to the formation 112 and thus the sensing member 118 will remain in the unactuated state, and thus the sensor 114 will output a LOW signal, until the dial sleeve 106 rotates to a position in which a portion of the leading edge 112A of a second formation 112, adjacent to the first formation 112, abuts the sensing member 118 and urges the sensing member 118 from the unactuated state to the actuated state such that the sensor 114 again transitions from outputting a LOW signal to outputting a HIGH signal.

Rotation of the dial sleeve 106 in the first rotational direction X therefore causes the sensor 114 to generate an oscillating signal, for example, a square wave signal. The signal is input to the processor 115. The processor 115 may then use edge detection to determine the angular displacement of the dial sleeve 106 to determine the dosage dispensed from the medicament reservoir.

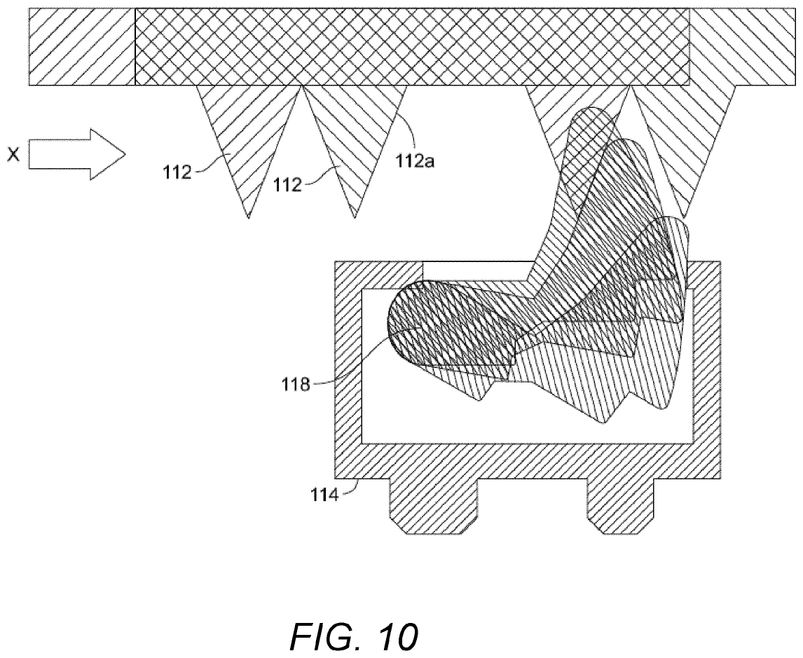

Movement of the sensing member 118 between the unactuated and actuated states is depicted in FIGS. 10 and 12, which show schematic representations of the sensor 114 and formations 112.

The dial sleeve 106 comprises a plurality of first and second encoding regions 123, 124 that are arranged alternately about the rotational axis of the dial sleeve 106. Each first encoding region 123 starts when the sensing member 118 is urged into the actuated state by the respective formation 112 abutting the sensing member 118 such that the sensor 114 transitions to outputting a HIGH signal, and ends with the formation 112 passing the sensor 114 such that the sensing member 118 moves back to the unactuated state and the sensor 114 transitions to outputting a LOW signal. Each second encoding region 124 starts at a rotational position of the dial sleeve 106 wherein the sensing member 118 reaches the actuated state in which the sensor 114 outputs a LOW signal, and ends with the sensing member 118 being urged into the unactuated state by an adjacent formation 112 abutting the sensing member 118 such that the sensor 114 outputs a HIGH signal.

The sensor 114 is thus able to distinguish between the first and second encoding regions 123, 124, because when the sensor 114 is aligned with a first encoding region 123 the sensing member 118 is in the unactuated state and the sensor 114 outputs a HIGH signal (or, in an alternative embodiment, a LOW signal) and when the sensor 114 is aligned with a second encoding region 124 the sensing member 118 is in the actuated state and the sensor 114 outputs a LOW signal (or, in said alternative embodiment, a HIGH signal).

Each first encoding region 123 subtends about the rotational axis of the dial sleeve 106 by a predetermined angle and each second encoding region 124 subtends about the rotational axis of the dial sleeve 106 by the same predetermined angle. This does not necessarily mean that the formations 112 subtend about the rotational axis of the dial sleeve 106 by the same angle as the gaps 113 between the formations 112. This is because the arrangement of the sensing member 118 and formation 112 is such that the sensing member 118 remains in the actuated state even once the apex 1128 of the formation 112 passes the apex 118A of the sensing member 118. For example, a trailing edge 112C of the formation 112, which faces generally in the opposite circumferential direction to the leading edge 112A, may still abut the sensing member 118 to prevent the sensing member 118 returning to the unactuated state. This is known as the ‘ON drag’, and refers to the relative movement of the engaged formation 112 and the sensor 114 required once the sensing member 118 has transitioned to the actuated state before the sensing member again transitions to the unactuated state. In one embodiment, the formations 112 and sensing member 118 are arranged such that the engaged formation 112 must move 1 mm relative to the sensing member 118 before the sensing member 118 transitions back to the unactuated state. The sensing member 118 stays in the actuated state for 1 mm of travel of the formation 112 once engaged and moved to the actuated state by the formation 112.

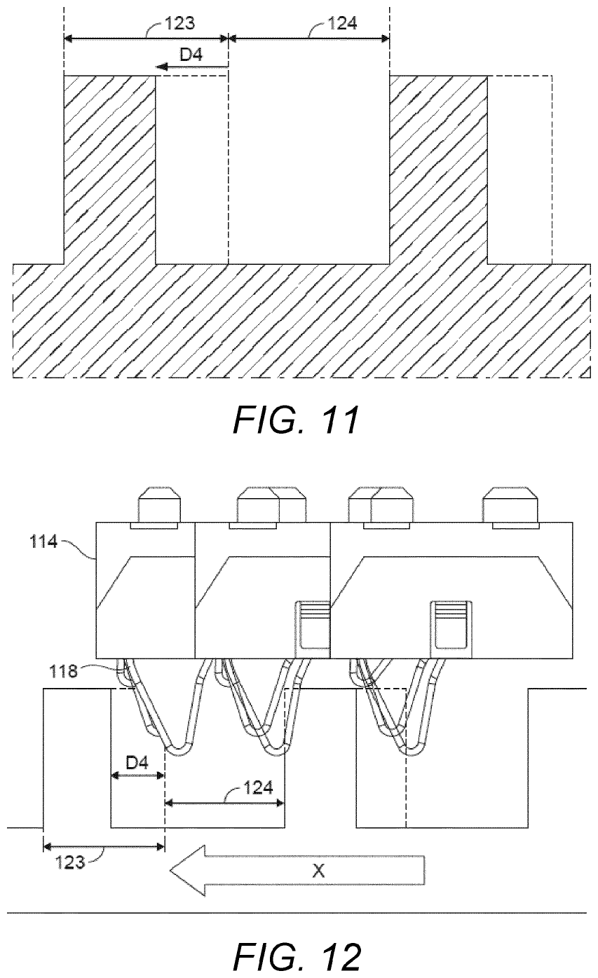

In some embodiments, the size and/or shape of each formation 112 is adjusted to take account of the ON drag such that the first and second encoding regions 123, 124 remain equal in size. For example, the angle that each formation 112 subtends about the rotational axis of the dial sleeve 106 may be reduced by the size of the ON drag (shown by arrow D4 in FIGS. 11 and 12).

In some alternative embodiments (not shown), the sensor 114 outputs a HIGH signal when the sensing member 118 is in the unactuated state and a LOW signal when in the actuated state. In yet further embodiments, the sensor 114 outputs an analogue signal that depends on the position of the sensing member 118, for example, a signal that is below a predetermined value when the sensing member 118 is in the unactuated state and above the predetermined value when in the actuated state. In some embodiments, the signal is generally sinusoidal as the dial sleeve 106 is rotated in the first rotational direction X.

In one embodiment, the processor 115 is configured to determine the dosage dispensed from the reservoir by counting the number of transitions from LOW to HIGH and the number of transitions from HIGH to LOW of the signal output by the sensor 114. In some embodiments, this involves edge counting of the signal generated by the sensor 114, for example, positive and/or negative edge counting.

The processor 115 is therefore able to determine the rotational displacement of the dial sleeve 106 during the delivery of medicament with a resolution equal to the angle that each of the first and second encoding regions 123, 124 subtends about the rotational axis of the dial sleeve 106. From this rotational displacement, the processor 115 can determine the amount of medicament dispensed from the reservoir. The smaller the angle that each of the first and second encoding regions 123, 124 subtends about the rotational axis, the greater the resolution of the dosage determination measurement.

The processor 115 may be configured to transmit the determined medicament dosage and, where determined, the time stamp information to another device, such as a computer (not shown). As noted above, the output 117 may be configured to transmit the information using a wireless communications link. Alternatively, the dosage measurement system 101 may be connected to the computer (not shown) using a wired connection (not shown) to allow the information to be uploaded to the computer. The processor 115 may be configured to transmit the information to the computer periodically. In some embodiments, the dosage measurement system 115 may be used to monitor compliance with a particular dosage regime.

In the present embodiment, the dial sleeve 106 comprises twenty-four formations 112. Thus, twenty-four first encoding regions 123 and twenty-four second encoding regions 124 are provided. Each encoding region 123, 124 subtends about the rotational axis of the dial sleeve 106 by an angle of 7.5 degrees.

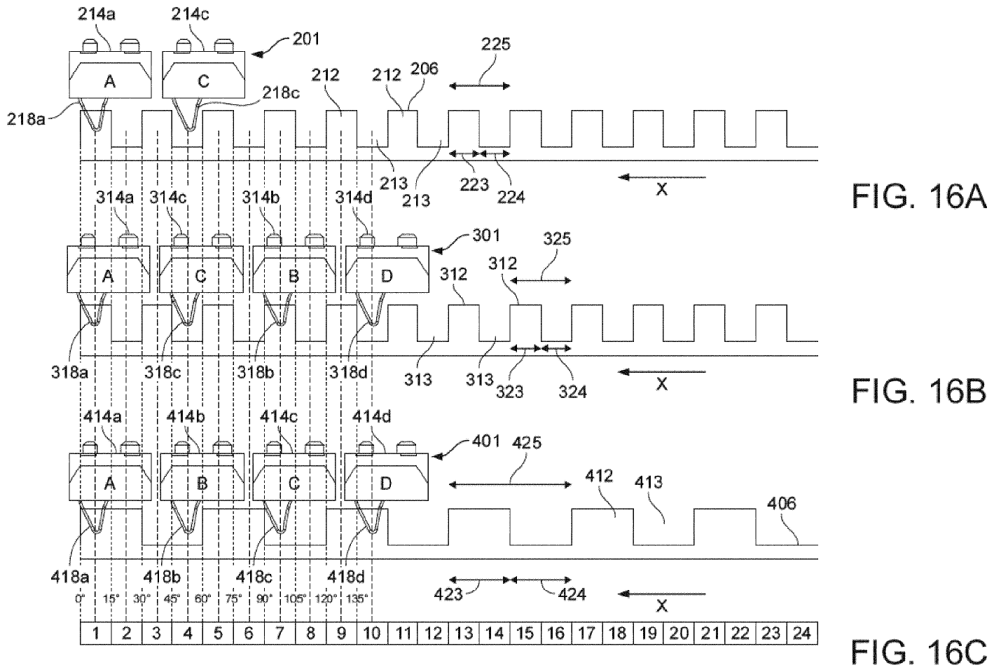

Referring now to FIGS. 16A to 18, another embodiment of a dosage measurement system 201 is shown. The dosage measurement system 201 is similar to the dosage measurement system 101 described above in relation to the embodiment of FIGS. 2 to 15, with like features retaining the same reference numerals, and is part of a medicament delivery device (not shown) having the same features as that of FIGS. 2 to 15. However, the dosage measurement system 201 is different in that it has first and second sensors 214A, 214C. The dial sleeve 206 comprises a plurality of formations 212 separated by gaps 213, wherein the formations 212 are configured to engage the first and second sensors 214A, 214C as the dial sleeve 206 is rotated.

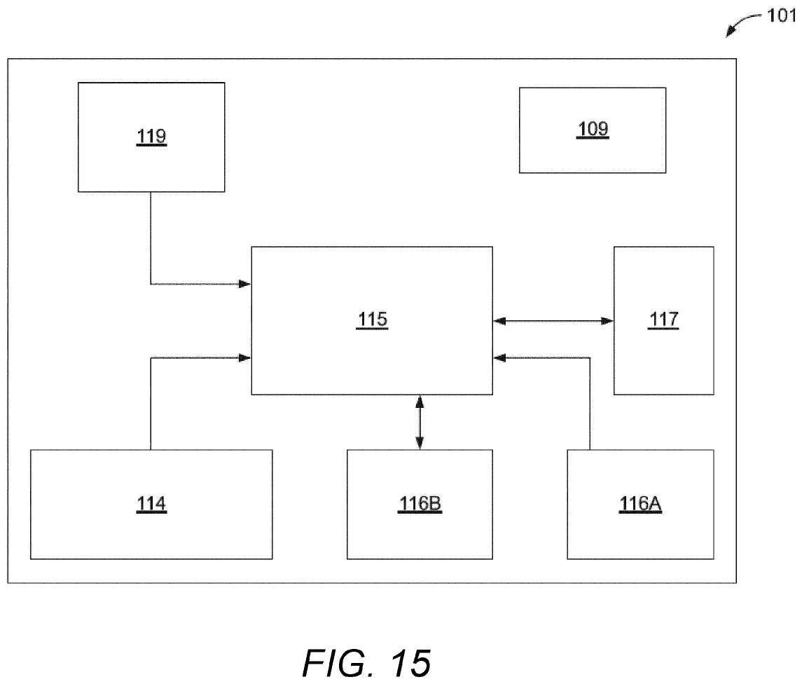

Each of the first and second sensors 214A, 214C may be of the same type as that of the sensor 114 of the embodiment described above in relation to FIGS. 2 and 15, having a sensing member 218A, 218C that is moveable between unactuated and actuated states, and thus a detailed description will not be repeated hereinafter.

As with the embodiment of FIGS. 2 and 15, the dial sleeve 206 comprises a plurality of first and second encoding regions 223, 224 that are arranged alternately about the rotational axis of the dial sleeve 206. The first encoding regions 223 are formed by the portions of respective formations 212 that urge the sensing members 218A, 218C to the actuated state when aligned therewith. The second encoding regions 224 are formed between the first encoding regions 223.

Each of the first and second sensors 214A, 214C generates a HIGH signal when the sensor 214A, 214C is aligned with one of the first encoding regions 223 and a LOW signal when aligned with one of the second encoding regions 224. Thus, each sensor 214A, 214C is able to distinguish between the first and second encoding regions 223, 224 and generate a signal accordingly.

The first and second sensors 214A, 214C are offset such that, in use, rotation of the dial sleeve 206 in the first rotational direction X causes one of the first encoder regions 223 to align with the first sensor 214A whilst one of the second encoder regions 224 aligns with the second sensor 214C. Moreover, further rotation of the dial sleeve 206 in the first rotational direction X causes one of the second encoder regions 224 to align with the first sensor 214A whilst one of the first encoder regions 223 aligns with the second sensor 214C. Thus, whilst the first sensor 214A detects one of the first encoding regions 223, the second sensor 214C will be aligned with one of the second encoding regions 224, and whilst the second sensor 214C detects one of the first encoding regions 223, the first sensor 214A will be aligned with one of the second encoding regions 224.

The relative positions of the first and second sensors 214A, 214C with respect to the first and second encoding regions 223, 224 are depicted in the schematic diagram of FIG. 16A. In FIG. 16A, the first sensor 214A is aligned with a first encoding region 223 and thus the first sensing member 218A would be in the actuated state, but this is not shown in order to give a clearer indication of the respective positions of the sensors 214A, 214C relative to the encoding regions 223, 224.

In FIG. 16A, the formations 212 and gaps 213 are shown schematically for illustrative purposes, FIG. 16A being provided to show the arrangement of the first and second encoding regions 223, 224 relative to the sensors 214A, 214C.

The first and second sensors 214A, 214C may be arranged such that for all rotational positions of the dial sleeve 206 in which the first sensor 214A aligns with one of the first encoding regions 223, the second sensor 214C aligns with one of the second encoding regions 224. Thus, when the sensing member 218A of the first sensor 214A transitions from the unactuated state to the actuated state during rotation of the dial sleeve 206, the sensing member 218C of the second sensor 214C transitions from the actuated state to the unactuated state.

In some embodiments, the first and second sensors 214A, 214C are offset by 165 degrees about the rotational axis of the drive sleeve 206 in the first rotational direction X.

The first and second sensors 214A, 214C may be arranged such that for all rotational positions of the dial sleeve 206 in which the first sensor 214A aligns with one of the second encoding regions 224, the second sensor 214C aligns with one of the first encoding regions 223. Thus, when the sensing member 218A of the first sensor 214A transitions from the actuated state to the unactuated state during rotation of the dial sleeve 206, the sensing member 218C of the second sensor 214C transitions from the unactuated state to the first second.

The processor (not shown) is configured to determine the delivered dose based on the movement of the first and second sensing members 218A, 218C. For example, the processor may be configured to count the number of times that the sensing members 218A, 218C transition from the unactuated state to the actuated state and/or the number of times that the sensing members 218A, 218C transition from the actuated state to the unactuated state. The processor may be pre-programmed with information allowing the processor to convert the number of transitions into a determination of the dosage delivered by the medicament delivery device.

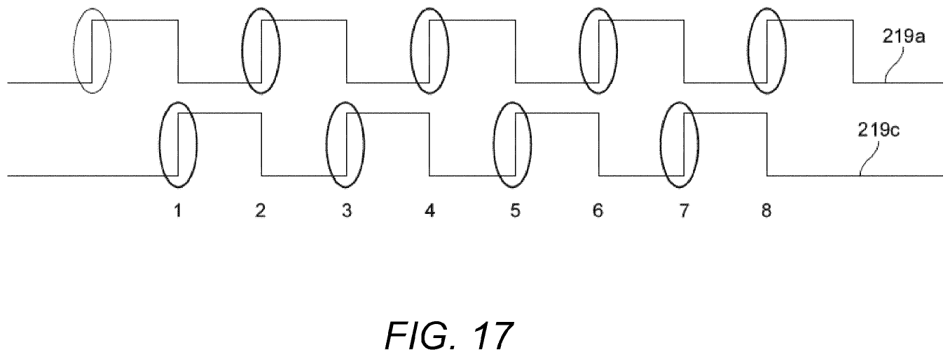

In one embodiment, the processor is configured to determine the delivered dose based on one of: counting the transitions of the sensing members 218A, 218C from the unactuated state to the actuated state; or, counting the transitions of the sensing members 218A, 218C from the second positon to the unactuated state. One such example is shown in FIG. 17, wherein the processor is configured to count the number of times that the signals 219A, 219C output from the first and second sensors 214A, 214C respectively transition from LOW to HIGH. This may be referred to as positive edge counting. In another example, the processor is configured to count the number of times that the signals 219A, 219C output from the first and second sensors 214A, 214C transition from HIGH to LOW. This may be referred to as negative edge counting.

The provision of first and second sensors 214A, 214C that are offset in the first rotational direction X advantageously means that resolution of the dosage determination is increased in comparison to embodiments that have only a single sensor. In the present embodiment, the resolution for a given size of formation 212 is doubled in comparison to embodiments that have only a single sensor. Therefore, the size of the formations 212 and thus the angle of each of the first and second encoding regions 223, 224 can be increased, resulting in larger edge tolerance as explained below, whilst achieving the same resolution as a single sensor configuration. For example, if first and second sensors 214A, 214C are used then the number of formations 212 can be reduced to twelve whilst achieving the same measurement resolution as the twenty-four formation 112 embodiment of FIGS. 2 to 15.

The provision of twelve formations 212 means that each of the first and second encoding regions 223, 224 subtends for an angle of 15 degrees about the rotational axis of the dial sleeve 206. If only a single sensor 114 was utilised, and the processor counted only one of the negative or positive edges, then the resolution of measurement would be 30 degrees, which is the angle that the dial sleeve 206 would need to rotate for successive transitions of HIGH to LOW (or LOW to HIGH in the negative edge counting embodiment) of the signal output by the sensor. However, since first and second sensors 214A, 214C are used, the resolution is doubled such that it is the same as a single sensor embodiment that has twenty four formations. However, the advantage of using fewer, larger, formations 212 (for instance, the twelve formations 212 in the present embodiment, although a skilled person would recognise that a different number of formations 212 could be used) is that a larger edge tolerance is achieved.

The edge tolerance is the maximum distance that the dial sleeve 206 must rotate (in either of the first or second rotational directions X, Y) for either sensor 214A, 214C to align with the transition between one of the first encoding regions 223 and one of the second encoding regions 224. Thus, in the present embodiment, with twelve formations 212 the maximum edge tolerance is 7.5 degrees. For comparison, in the embodiment with a single sensor 114 and twenty-four formations 112, the edge tolerance is 3.75 degrees.

A larger edge tolerance is advantageous because it reduces the chance of a false edge detection by the sensors 214A, 214C, which may otherwise occur due to mechanical tolerances. This is because for embodiments with a larger edge tolerance the sensing members 218A, 218C are less likely to transition from one of the unactuated and actuated states to the other one of the first and second positons for a given rotational displacement of the dial sleeve 206. Thus, for example, a small amount of ‘play’ or accidental rotation of the dial sleeve 206 relative to the housing 202 is less likely to cause the first and second sensors 214A, 214C to transition between outputting LOW/HIGH signals, which would otherwise result in a false edge detection by the processor and thus a measurement error.

In some embodiments, the edge tolerance is at least 5 degrees, at least 7 degrees and, preferably, the edge tolerance is at least 7.5 degrees.

In some embodiments, the first and second sensors 214A, 214C are mounted to a dose setting dial (not shown) such that the sensors 214A, 214C are moveable from an idle positon to a detecting position, in a similar manner to as described in relation to the embodiment of FIGS. 2 to 15.

The first and second sensors 214A, 214C may be arranged such that for all rotational positions of the dial sleeve 206, one of the first and second sensors 214A, 214C aligns with a first encoding region 223 and the other one of the first and second sensors 214A, 214C aligns with a second encoding region 224. This means that when the dose setting dial 208 (not shown) is actuated by the user such that the first and second sensors 214A, 214C move to the detecting position, one of the first and second sensors 214A, 214C will engage with one of the formations 212 such that the sensing member 218A, 218C of said sensor 214A, 214C moves to the actuated state and thus the signal output from said sensor 214A, 214C transitions from LOW to HIGH (or the other sensor outputs such a transition in embodiments where the sensors output inverse signals).

If this transition was counted and used to determine the dosage dispensed from the reservoir, then the determined dosage would be larger than the actual dosage dispensed. To compensate for this, in some embodiments the processor is configured to disregard the first detected LOW to HIGH transition that is output from the sensors 214A, 214C since this is caused by the axial movement of one the sensors 214A, 214C to the detecting position. The remaining transitions from LOW to HIGH are taken into account by the processor to determine the dosage dispensed, since these transitions are a result of rotation of the dial sleeve 206 during the dispensing of medicament rather than being due to axial movement of the sensors 214A, 214C.

In some embodiments, the dial sleeve 206 comprises a plurality of encoding periods 225, wherein each encoding period 225 comprises one of the first encoding regions 223 and an adjacent second encoding region 224. In some embodiments, for a given rotational position of the dial sleeve 206 the first sensor 214A is aligned with a portion of one of the encoding periods 225 and the second sensor 214C is aligned with a different portion of one of the encoding periods 225. If each encoding period 225 is taken to have an imaginary period of 360 degrees, that is, the formations 212 repeat every 360 degrees, then the first and second sensors 214A, 214C may be offset by 180 degrees of the encoding period 225.

In some embodiments, the offset angle of the first and second sensors 214A, 214C about the rotational axis of the dial sleeve 206 is an odd number integer multiple of the angle that each first encoding region 223 subtends about the rotational axis of the drive sleeve 206. For instance, if the first encoding region 223 subtends about the rotational axis by 15 degrees, then the offset angle may be 15, 45, 75, 105, 135, 165, 195, 225, 255, 285, 315 or 345 degrees.

In one such embodiment, the first and second sensors 214A, 214C are offset by an angle that is eleven times the angle that each first encoding region 223 subtends about the rotational axis of the drive sleeve 206. The first and second sensors 214A, 214C are offset by 165 degrees about the rotational axis of the drive sleeve 206.

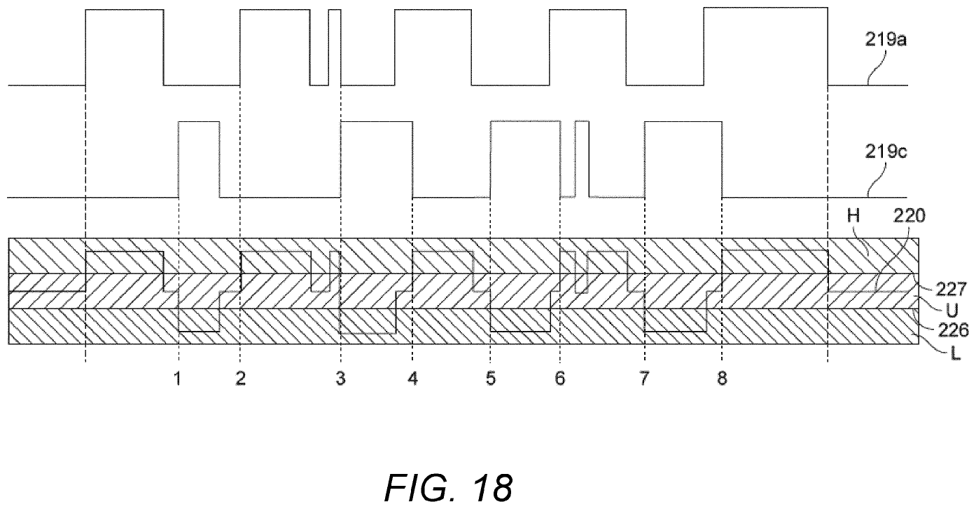

The processor may be configured to determine a dosage dispensed from the medicament reservoir based on a signal output from one of the first and second sensors 214A, 214C and an inverse of the signal output from the other one of the first and second sensors 214A, 214C. In one embodiment shown in FIG. 18, the processor is configured to determine a dosage dispensed from the medicament reservoir based on a superposition of the signal from one of the first and second sensors 214A, 214C and the inverse signal from the other one of the first and second sensors 214A, 214C. In this particular embodiment, a superposition 220 of the signal 219A output from the first sensor 214A and the inverse of the signal 219C output from the second sensor 214C.

The superposition 220 may be calculated by adding the first signal 219A and the inverse of the second signal 219C.

The processor is configured to determine the dosage dispensed from the medicament reservoir by comparing the superposition 220 to a first threshold value 226 and a second threshold value 227, greater than the first threshold value 226. In one such embodiment, the processor counts the number of times the superposition 220 transitions from a value lower than the first threshold value 226 to a value greater than the second threshold value 227 and/or the number of times the superposition transitions from a value higher than the second threshold value 227 to a value lower than the first threshold value 226. This helps to improve the accuracy of the dosage determination by helping to filter out errors such as sensor noise and switch bouncing. This is because if one of the first and second sensors 214A, 214C gives an erroneous reading then this will not cause the superposition 220 to transition in a manner that is counted by the processor in the dosage determination calculation.

In the present embodiment, the processor counts the number of times the superposition 220 transitions from a value lower than the first threshold value 226 to a value greater than the second threshold value 227 and also the number of times the superposition transitions from a value higher than the second threshold value 227 to a value lower than the first threshold value 226. This count is used to determine the dosage dispensed from the reservoir. The first count may be disregarded in embodiments with axially moving sensors 214A, 214C on an actuator, as described previously.

The first threshold value 226 may be such that when the first sensor 214A is aligned with a second encoding portion 224 and the second sensor 214C is simultaneously aligned with a first encoding portion 223, the superposition 220 is below the first threshold value 226 and otherwise is above the first threshold value 226. That is, for the superposition 220 to be below the first threshold value 226, the first signal 219A and the inverse of the second signal 219C both have to be LOW.

The second threshold value 227 may be such that when the first sensor 214A is aligned with a first encoding portion 223 and the second sensor 214C is simultaneously aligned with a second encoding portion 224, the superposition 220 is above the second threshold value 227 and otherwise is below the second threshold value 227. That is, for the superposition 220 to be above the second threshold value 227, the first signal 219A and the inverse of the second signal 219C both have to be HIGH.

In some embodiments, when the superposition 220 is equal to or below the first threshold value 226 the superposition 220 is in a LOW state L and when the superposition 220 is equal to or above the second threshold value 227 the superposition 220 is in a HIGH state H. When the superposition 220 is above the first threshold value 226 but below the second threshold value 227 the superposition 220 is in an UNDEFINED state. The processor may count the number of times the superposition 220 transitions from LOW to HIGH states and/or from HIGH to LOW states to determine the dispensed dosage.

For example, for certain rotational positions of the dial sleeve 206, the signal 219A output from the first sensor 214A is supposed to be HIGH and the signal 219C output from the second sensor 214C is supposed to be LOW such that the inverse of the signal 219C output from the second sensor 214C is supposed to be HIGH and thus both HIGH signals mean that the superposition signal 220 is supposed to be above the second threshold value 227 (the superposition 220 is supposed to be in a HIGH state H). However, the mechanical switch of the first sensor 214A may momentarily ‘bounce’ such that it outputs a LOW reading. In such a scenario, the superposition 220 would be below the second threshold value 227 but would still be greater than the first threshold value 226 because the inverse signal is still HIGH (the superposition would be in the UNDEFINED state). The superposition signal 220 would only transition to a value lower than the first threshold value 226, and thus be counted by the processor to determine the dispensed dosage, when both the signal 219A output from the first sensor 214A and the inverse of the signal 219C output from the second sensor 214C are both LOW (at which point the superposition 220 would transition to the LOW state L, the total transition from HIGH to LOW being counted as one increment by the processor in the dispensed dosage determination). Thus, the switch bounce will not be counted by the processor in the dosage dispensing calculation and thus will not contribute to an erroneous dosage determination value. Alternatively, if after the ‘bounce’ the first sensor 214A instead again output a HIGH reading, the superposition 220 would transition back to the High state H and this would not be counted as an increment by the processor because the superposition was previously in HIGH state H before moving to the UNDEFINED state and thus had not transitioned from the LOW state L to the HIGH state H.

In some embodiments, when the dial sleeve 206 has reached the zero position and has finished rotation such that the programmed dosage has been dispensed from the reservoir, the first and second sensors 214A, 214B will each align with a particular encoding region 223, 224 for longer than when the dial sleeve 206 is rotating and the first and second sensors 214A,214C are aligning with successive encoding regions 223, 224. This is illustrated in FIG. 18, which shows that the signals 219A, 219C output from the first and second sensors 223, 224 remain in HIGH and LOW states (and thus the superposition 220 remains in the HIGH state) for a longer time period after the last transition between first and second encoding regions 223, 224 detected by the sensors 214A, 214C during rotation of the dial sleeve 206 has occurred (transition number 8 in the example of FIG. 18). In FIG. 18, the final transition after transition number 8 is due to the actuator, in the present case the dose setting dial 208, moving proximally away from its actuated position such that the sensors 214A, 214C move out of the detecting positions and back to the idle positions and thus no longer engage the formations 212.

The processor may therefore determine that the dial sleeve 206 has stopped rotating, and thus the dosage has been dispensed, if the first sensor 214A and/or second sensor 214C remains aligned with one of the first and second encoding regions 223, 224 for more than a predetermined amount of time. The predetermined amount of time may be selected to be greater than the amount of time that any of the first and second encoding regions 223, 224 would remain aligned with one of the first and second sensors 214A, 214C during rotation of the dial sleeve 206 during operation of the dispensing mechanism to dispense medicament from the reservoir.

If the processor determines that the dosage has been dispensed, the processor may perform one or more operations, for example: indicating to the user that a dosage has been dispensed, for example, by a user interface such as an LED, speaker or screen or by transmitting a signal to display such information on the separate device; storing and/or transmitting data relating to the dispensed dosage; disregarding any further transitions between first and second encoding regions detected by the sensors such that said further transitions are not used to calculate the dosage dispensed; and/or, powering down the dosage measurement system to conserve energy.

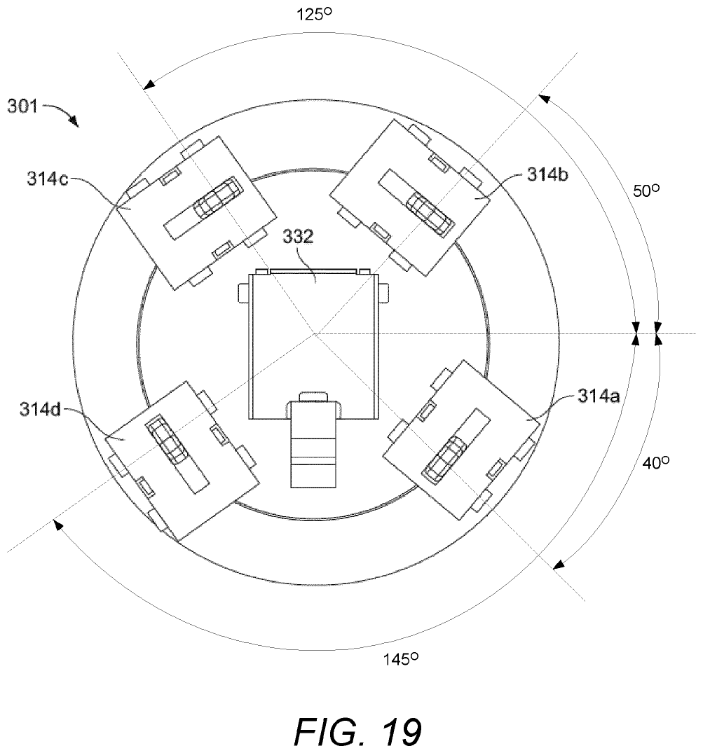

Referring now to FIGS. 16B, 19 and 20, another embodiment of a dosage measurement system 301 is shown. The dosage measurement system 301 is similar to the dosage measurement system 201 described above in relation to the embodiment of FIGS. 16A and 17 to 18, with like features retaining the same reference numerals, and is part of a medicament delivery device (not shown) having the same features as that of FIGS. 2 to 15. However, the dosage measurement system 301 is different in that, in addition to comprising first and second sensors 314A, 314C, the dosage measurement system 301 further comprises third and fourth sensors 314B, 314D.

The dial sleeve 306 comprises a plurality of formations 312 separated by gaps 313, wherein the formations 312 are configured to engage the first, second, third and fourth sensors 314A, 314C, 314B, 314D as the dial sleeve 306 is rotated.

Each of the first, second, third and fourth sensors 314A, 314C, 314B, 314D may be of the same type as the first and second sensors 214A, 214C of the embodiment described above in relation to FIGS. 16A and 17 to 18, having first, second, third and fourth sensing members 318A, 318C, 318B, 318D respectively that are each moveable between unactuated and actuated states, and thus a detailed description will not be repeated hereinafter.

As with the embodiment of FIGS. 16A and 17 to 18, the dial sleeve 306 comprises a plurality of first and second encoding regions 323, 324 that are arranged alternately about the rotational axis of the dial sleeve 306. The first encoding regions 323 are formed by the portions of respective formations 312 that urge the sensing members 318A, 318C, 318B, 318D to the actuated state when aligned therewith. The second encoding regions 324 are formed between the first encoding regions 323.

Each of the first, second, third and fourth sensors 314A, 314C, 314B, 314D generates a HIGH signal when the sensor 314A, 314C, 314B, 314D is aligned with one of the first encoding regions 323 and a LOW signal when aligned with one of the second encoding regions 324. Thus, each sensor 314A, 314C, 314B, 314D is able to distinguish between the first and second encoding regions 323, 324 and generate a signal accordingly.

As with the embodiment of the dosage measurement system 201 of FIGS. 16A and 17 to 18, the first and second sensors 314A, 314C are offset such that, in use, rotation of the dial sleeve 306 in the first rotational direction X causes one of the first encoder regions 323 to align with the first sensor 314A whilst one of the second encoder regions 324 aligns with the second sensor 314C. Moreover, further rotation of the dial sleeve 306 in the first rotational direction X causes one of the second encoder regions 324 to align with the first sensor 314A whilst one of the first encoder regions 323 aligns with the second sensor 314C. Thus, whilst the first sensor 314A detects one of the first encoding regions 323, the second sensor 314C will be aligned with one of the second encoding regions 324, and whilst the second sensor 314C detects one of the first encoding regions 323, the first sensor 314A will be aligned with one of the second encoding regions 324.

The third and fourth sensors 314B, 314D are offset from the first and second sensors 314A, 314C in the direction of rotation of the dial sleeve 306.

In some embodiments, the first and second sensors 314A, 314C are offset by 165 degrees about the rotational axis of the drive sleeve 306 in the first rotational direction X. In some embodiments, third sensor 314B, is offset from the first sensor 314A about the rotational axis in the first rotational direction X by 90 degrees and wherein the fourth sensor 314D is offset from the first sensor 314A about the rotational axis in the second rotational direction by 105 degrees.

In FIG. 16B, the formations 312 and gaps 313 are shown schematically for illustrative purposes, FIG. 16B being provided to show the arrangement of the first and second encoding regions 323, 324 relative to the sensors 314A, 314B, 314C, 314D.

The third sensor 314B is arranged such that, in use, rotation of the dial sleeve 306 in the first rotational direction X causes one of the first encoder regions 323 to align with the first sensor 314A whilst another one of the first encoder regions 323 aligns with the third sensor 314B. Moreover, further rotation of the dial sleeve 306 in the first rotational direction X causes one of the second encoder regions 324 to align with the first sensor 314A whilst another one of the second encoder regions 323 aligns with the third sensor 314B. Thus, whilst the first sensor 314A detects one of the first encoding regions 323, the third sensor 314B will be aligned with another one of the first encoding regions 323, and whilst the first sensor 314A detects one of the second encoding regions 324, the third sensor 314B will be aligned with another one of the second encoding regions 324.

The third sensor 314B may be arranged such that for all rotational positions of the dial sleeve 306 in which the first sensor 314A aligns with one of the first encoding regions 323, the third sensor 314B aligns with one of the first encoding regions 323. Thus, when the sensing member 318A of the first sensor 314A transitions from the unactuated state to the actuated state during rotation of the dial sleeve 306, the sensing member 318B of the third sensor 314B transitions from the unactuated state to the actuated state. Similarly, the third sensor 314B may be arranged such that for all rotational positions of the dial sleeve 306 in which the first sensor 314A aligns with one of the second encoding regions 324, the third sensor 314B aligns with one of the second encoding regions 324. Thus, when the sensing member 318A of the first sensor 314A transitions from the actuated state to the unactuated state during rotation of the dial sleeve 306, the sensing member 318B of the third sensor 314B transitions from the actuated state to the unactuated state

The fourth sensor 314D is arranged such that, in use, rotation of the dial sleeve 306 in the first rotational direction X causes one of the first encoder regions 323 to align with the second sensor 314C whilst another one of the first encoder regions 323 aligns with the fourth sensor 314D. Moreover, further rotation of the dial sleeve 306 in the first rotational direction X causes one of the second encoder regions 324 to align with the second sensor 314C whilst another one of the second encoder regions 323 aligns with the fourth sensor 314D. Thus, whilst the second sensor 314C detects one of the first encoding regions 323, the fourth sensor 314D will be aligned with another one of the first encoding regions 323, and whilst the second sensor 314C detects one of the second encoding regions 324, the fourth sensor 314D will be aligned with another one of the second encoding regions 324.

The fourth sensor 314D may be arranged such that for all rotational positions of the dial sleeve 306 in which the second sensor 314C aligns with one of the first encoding regions 323, the fourth sensor 314D aligns with one of the first encoding regions 323. Thus, when the sensing member 318C of the second sensor 314C transitions from the unactuated state to the actuated state during rotation of the dial sleeve 306, the sensing member 318D of the fourth sensor 314D transitions from the unactuated state to the actuated state. Similarly, the fourth sensor 314D may be arranged such that for all rotational positions of the dial sleeve 306 in which the second sensor 314C aligns with one of the second encoding regions 324, the fourth sensor 314D aligns with one of the second encoding regions 324. Thus, when the sensing member 318C of the second sensor 314C transitions from the actuated state to the unactuated state during rotation of the dial sleeve 306, the sensing member 318D of the fourth sensor 314D transitions from the actuated state to the unactuated state.

In some embodiments, the dial sleeve 306 comprises a plurality of encoding periods 325, wherein each encoding period 325 comprises one of the first encoding regions 323 and an adjacent second encoding region 324. In some embodiments, for a given rotational position of the dial sleeve 306 the first sensor 314A is aligned with a portion of one of the encoding periods 325 and the second sensor 314C is aligned with a different portion of one of the encoding periods 325. If each encoding period 325 is taken to have an imaginary period of 360 degrees, that is, the formations 312 repeat every 360 degrees, then the first and second sensors 314A, 314C may be offset by 180 degrees of the encoding period 325.

The third sensor 314B is in phase with the first sensor 314A such that the sensors 314B, 314A are aligned with the same portions of respective encoding periods 325 and thus are offset by zero degrees of the encoding period 325. The fourth sensor 314D is in phase with the second sensor 314C such that the sensors 314D, 314C are aligned with the same portions of respective encoding periods 325 and thus are offset by zero degrees of the encoding period 325.

The third sensor 314B provides redundancy should the first sensor 314A fail to detect the encoding region 323, 324 due to a detecting error and also allows for improbable detection events to be filtered. The fourth sensor 314D provides redundancy should the second sensor 314C fail to detect the encoding region 323, 324 due to a detecting error and also allows for improbable detection events to be filtered [please explain examples of such improbable detection events].

The processor may be configured to determine a dosage dispensed from the medicament reservoir based on a superposition of the signals 319A, 319B output from the first and third sensors 314A, 314B and the inverse of the signals 319C, 319D output from the second and fourth sensors 314C, 314D, or, a superposition of the signals 319C, 319D output from the second and fourth sensors 314C, 314D and the inverse of the signals 319A, 319B output from the first and third sensors 314A, 314B.

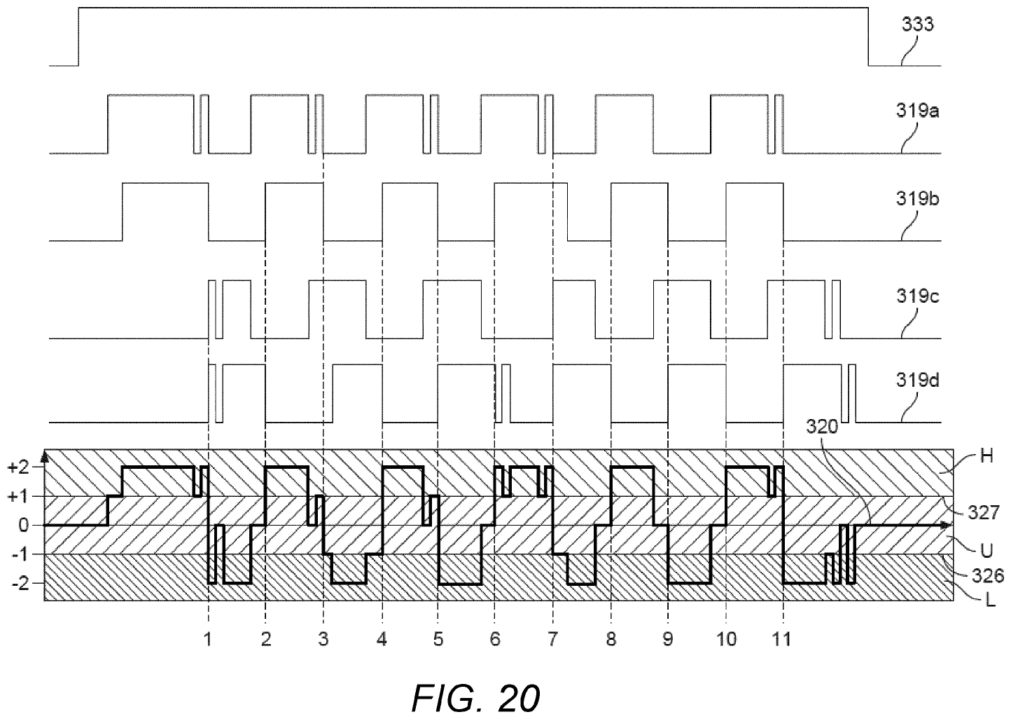

In the particular embodiment shown in FIG. 20, the dosage is determined based on a superposition 320 of the signals 319A, 319B output from the first and third sensors 314A, 314B and the inverse of the signals 319C, 319D output from the second and fourth sensors 314C, 314D

The superposition 320 may be calculated by adding the first and third signals 219A, 219B with the inverse of the second and fourth signals 219C, 219D.

The processor is configured to determine the dosage dispensed from the medicament reservoir by comparing the superposition 320 to a first threshold value 326 and a second threshold value 327, greater than the first threshold value 326.

In one such embodiment, the processor counts the number of times the superposition 320 transitions from a value lower than the first threshold value 326 to a value greater than the second threshold value 327 and/or the number of times the superposition transitions from a value higher than the second threshold value 327 to a value lower than the first threshold value 326. This helps to improve the accuracy of the dosage determination by helping to filter out errors such as sensor noise and switch bouncing. This is because if one or two of the sensors 314A, 314B, 314C, 314D gives an erroneous reading then this will not cause the superposition 320 to transition in such a manner that is counted by the processor in the dosage determination calculation.

In the present embodiment, the processor counts the number of times the superposition 220 transitions from a value lower than the first threshold value 326 to a value greater than the second threshold value 327 and also the number of times the superposition transitions from a value higher than the second threshold value 327 to a value lower than the first threshold value 326. This count is used to determine the dosage dispensed from the reservoir. The first count may be disregarded in embodiments with axially moving sensors 314A, 314B, 314C, 314D on an actuator that operates an activation switch 332, as described previously. The activation switch 332 signal 333 is shown in FIG. 20.

The first threshold value 326 may be such that for the superposition 320 to be below the first threshold value 326 at least three of: the signals 319A, 319B output from the first and third sensors 314A, 314B and the inverse of the signals 319C, 319D output from the second and fourth sensors 314C, 314D, must be LOW.

The second threshold value 227 may be such that for the superposition 320 to be above the second threshold value 326 at least three of: the signals 319A, 319B output from the first and third sensors 314A, 314B and the inverse of the signals 319C, 319D output from the second and fourth sensors 314C, 314D, must be HIGH.

In some embodiments, when the superposition 320 is equal to or below the first threshold value 326 the superposition 320 is in a LOW state L and when the superposition 320 is equal to or above the second threshold value 327 the superposition 320 is in a HIGH state H. When the superposition 320 is above the first threshold value 326 but below the second threshold value 327 the superposition 320 is in an UNDEFINED state. The processor may count the number of times the superposition 320 transitions from LOW to HIGH states and/or from HIGH to LOW states to determine the dispensed dosage.

For example, for certain rotational positions of the dial sleeve 306, the signals 319A, 319B output from the first and third sensors 314A, 314C are supposed to be HIGH and the inverse of the signals 319C, 319D output from the second and fourth sensors 314B, 314D are supposed to be HIGH and thus four HIGH signals mean that the superposition signal 320 is supposed to be equal to or above the second threshold value 327 (the superposition 320 is supposed to be in a HIGH state H). However, the mechanical switch of the first sensor 314A may momentarily ‘bounce’ such that it outputs a LOW reading. In such a scenario, the superposition 320 still be equal to or higher than the second threshold value 327 and thus the state of the superposition 320 will remain in the HIGH state H. The superposition 320 thus does not transition to the LOW state and so the processor does not count an increment for the dispensed dosage determination calculation. If, in fact, the mechanical switch of the fourth sensor 314D also simultaneously ‘bounces’ such that it outputs a HIGH reading and thus the inverse of the signal 319D is LOW, then the superposition 320 comprises two LOW and two HIGH signal inputs and thus the superposition 320 will be greater than the first threshold value 326 but less than the second threshold value 327. The superposition 320 will thus be in the UNDEFINDED state U.

The superposition signal 220 would only transition to a value equal to or lower than the first threshold value 226, and thus be counted by the processor to determine the dispensed dosage, when at least three of the following happens: the first sensor 314A outputs a signal 319A that is LOW, the third sensor 314B outputs a signal 319B that is LOW, the second sensor 314C outputs a signal 319C that is HIGH such that the inverse is LOW, and the fourth sensor 314D outputs a signal 319D that is HIGH such that the inverse is LOW (at which point the superposition 320 would transition to the LOW state L, the total transition from HIGH to LOW being counted as one increment by the processor in the dispensed dosage determination). Thus, the multiple switch bounce or other such errors will not be counted by the processor in the dosage dispensing calculation and thus will not contribute to an erroneous dosage determination value.

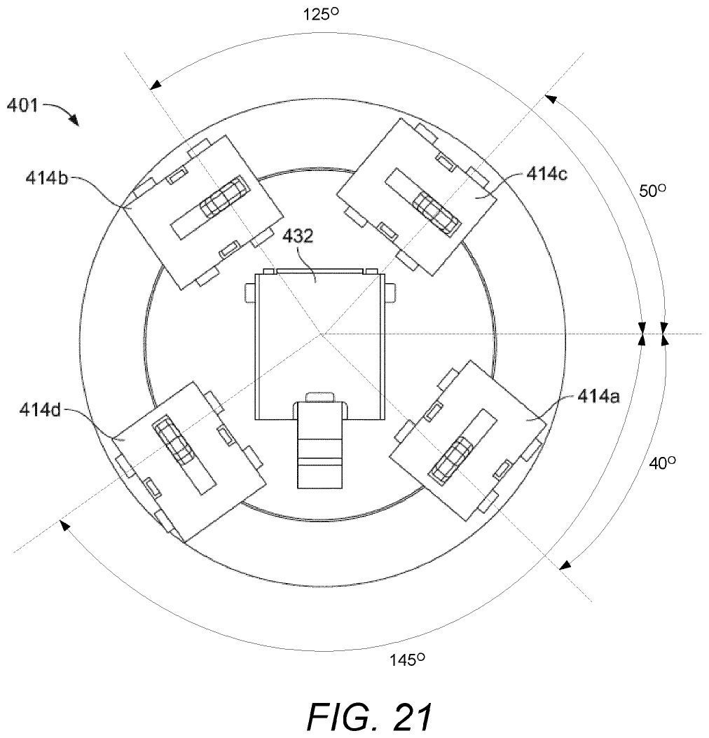

Referring not to FIGS. 16C and 21, another embodiment of a dosage measurement system 401 is shown. The dosage measurement system 401 of FIG. 16C is similar to the dosage measurement system 301 of the embodiment of FIGS. 16B, 19 and 20, with like features retaining the same reference numerals. A difference is that the formations 412 and gaps 413 of the dial sleeve 406 have a different sizing and the first, second, third and fourth sensors 414A, 414C, 414B, 414D have a different arrangement with respect to the first and second encoding regions 423, 424.

The dial sleeve 406 comprises half as many formation 412 and gaps 413 as the dial sleeve 306 of FIGS. 16B, 19 and 20. In the present embodiment, the dial sleeve 406 comprises six formations 412 and six gaps 413.

As with the embodiment of FIGS. 16A and 17 to 18 and the embodiment of FIGS. 16B and 19 to 20, the dial sleeve 406 comprises a plurality of first and second encoding regions 423, 424 that are arranged alternately about the rotational axis of the dial sleeve 406. The first encoding regions 423 are formed by the portions of respective formations 412 that urge the sensing members 418A, 418C, 418B, 418D to the actuated state when aligned therewith. The second encoding regions 424 are formed between the first encoding regions 423.

Each of the first, second, third and fourth sensors 414A, 414C, 414B, 414D generates a HIGH signal when the sensor 414A, 414C, 414B, 414D is aligned with one of the first encoding regions 423 and a LOW signal when aligned with one of the second encoding regions 424. Thus, each sensor 414A, 414C, 414B, 414D is able to distinguish between the first and second encoding regions 423, 424 and generate a signal accordingly.

As with the embodiment of the dosage measurement system 201 of FIGS. 16A and 17 to 18 and the embodiment of FIGS. 16B and 19 to 20, the first and second sensors 414A, 414C are offset such that, in use, rotation of the dial sleeve 406 in the first rotational direction X causes one of the first encoder regions 423 to align with the first sensor 414A whilst one of the second encoder regions 424 aligns with the second sensor 414C. Moreover, further rotation of the dial sleeve 406 in the first rotational direction X causes one of the second encoder regions 424 to align with the first sensor 414A whilst one of the first encoder regions 423 aligns with the second sensor 414C. Thus, whilst the first sensor 414A detects one of the first encoding regions 423, the second sensor 414C will be aligned with one of the second encoding regions 424, and whilst the second sensor 414C detects one of the first encoding regions 423, the first sensor 414A will be aligned with one of the second encoding regions 424.

The third and fourth sensors 414B, 414D are offset from the first and second sensors 414A, 414C in the direction of rotation of the dial sleeve 406. In FIG. 16C, the formations 412 and gaps 413 are shown schematically for illustrative purposes, FIG. 16C being provided to show the arrangement of the first and second encoding regions 423, 424 relative to the sensors 414A, 414B, 414C, 414D.

In some embodiments, the first and third sensors 414A, 414B are offset by 165 degrees about the rotational axis of the drive sleeve 406 in the first rotational direction X. In some embodiments, the second sensor 414C, is offset from the first sensor 414A about the rotational axis in the first rotational direction X by 90 degrees and wherein the fourth sensor 414D is offset from the first sensor 414A about the rotational axis in the second rotational direction by 105 degrees.

The third sensor 414B is arranged such that when the dial sleeve 406 is rotated such that, when the first sensor 414A is aligned with a transition between encoding regions 423, 424 and the second sensor 414C is aligned with a transition between encoding regions 423, 424, the third sensor 414B is not aligned with a transition between encoding regions 423, 424 (i.e. the third sensor 414B is only aligned with one of the first tor second encoding regions 423, 424). In some embodiments, when the dial sleeve 406 is in such a position, the third sensor 414B is equidistant from the two nearest transitions between first and second encoding regions 423, 424.

The fourth sensor 414D is arranged such that when the dial sleeve 406 is rotated such that, when the first sensor 414A is aligned with a transition between encoding regions 423, 424 and the second sensor 414C is aligned with a transition between encoding regions 423, 424, the fourth sensor 414D is not aligned with a transition between encoding regions 423, 424 (i.e. the fourth sensor 414D is only aligned with one of the first tor second encoding regions 423, 424). In some embodiments, when the dial sleeve 406 is in such a position, the fourth sensor 414D is equidistant from the two nearest transitions between first and second encoding regions 423, 424.

The third and fourth sensors 414B, 414D are offset such that, in use, rotation of the dial sleeve 406 in the first rotational direction X causes one of the first encoder regions 423 to align with the third sensor 414B whilst one of the second encoder regions 424 aligns with the fourth sensor 414D. Moreover, further rotation of the dial sleeve 406 in the first rotational direction X causes one of the second encoder regions 424 to align with the third sensor 414B whilst one of the first encoder regions 423 aligns with the fourth sensor 414D. Thus, whilst the third sensor 414B detects one of the first encoding regions 423, the fourth sensor 414D will be aligned with one of the second encoding regions 424, and whilst the fourth sensor 414D detects one of the first encoding regions 423, the third sensor 414B will be aligned with one of the second encoding regions 424.

In some embodiments, when the first sensor 414A is aligned with a transition between encoding regions 423, 424 the second sensor 414C is aligned with a transition between encoding regions 423, 424, and wherein when the third sensor 414B is aligned with a transition between encoding regions 423, 424 the fourth sensor 414D is aligned with a transition between encoding regions 423, 424.

In some embodiments, the dial sleeve 306 comprises a plurality of encoding periods 425, wherein each encoding period 425 comprises one of the first encoding regions 423 and an adjacent second encoding region 424. In some embodiments, for a given rotational position of the dial sleeve 406, the first, second, third and fourth sensors 414A, 414C, 414B, 414D are all each aligned with a different portion of a respective encoding period 425.

If each encoding period 425 is taken to have an imaginary period of 360 degrees, that is, the formations 412 repeat every 360 degrees, then the first and second sensors 414A, 414C may be offset by 180 degrees of the encoding period 425. The third and fourth sensors 414B, 414D may be offset by 180 degrees of the encoding period 425.

In some embodiments, the third sensor 414B may be offset by 90 degrees to one of the first and second sensors 414A, 414C and by 270 degrees to the other one of the first and second sensors 414A, 414C. The fourth sensor 414B may be offset by 270 degrees to said one of the first and second sensors 414A, 414C and by 90 degrees to said other one of the first and second sensors 414A, 414C.

In the present embodiment, the third sensor 414B is offset by 270 degrees to the first sensor 414A and by 90 degrees to the second sensor 414C. The fourth sensor 414D is offset by 90 degrees to the first sensor 414A and by 270 degrees to the second sensor 414C.

Since the first, second, third and fourth sensors 414A, 414C, 414B, 414D are all out of phase with respect to each other, the processor is able to determine whether the dial sleeve 406 is being rotated in the first or second rotational direction. Only one of the sensors 414A, 414C, 414B, 414D aligns with a positive edge transition from a first encoding region 423 to a second encoding region 424 at a time when the dial sleeve 406 is rotated.

For example, if the dial sleeve 406 is rotated in the first rotational direction X then actuation will be in the order of: the first sensor 414A, third sensor 414B, second sensor 414C and then the fourth sensor 414D. That is, one of the formations 212 will abut the sensing member 418A of the first sensor 414A to move the sensing member 418A from the unactuated state to the actuated state; one of the formations 212 will abut the sensing member 418B of the third sensor 414B to move the sensing member 418B from the unactuated state to the actuated state; one of the formations 212 will abut the sensing member 418C of the second sensor 414C to move the sensing member 418C from the unactuated state to the actuated state; and, then, one of the formations 212 will abut the sensing member 418D of the fourth sensor 414D to move the sensing member 418D from the unactuated state to the actuated state.

Conversely, if the dial sleeve 406 is rotated in the second rotational direction then actuation will be in the order of: the first sensor 414A, fourth sensor 414D, second sensor 414C and then the third sensor 414B. That is, one of the formations 212 will abut the sensing member 418A of the first sensor 414A to move the sensing member 418A from the unactuated state to the actuated state; one of the formations 212 will abut the sensing member 418D of the fourth sensor 414D to move the sensing member 418D from the unactuated state to the actuated state; one of the formations 212 will abut the sensing member 418C of the second sensor 414C to move the sensing member 418C from the unactuated state to the actuated state; and, then, one of the formations 212 will abut the sensing member 418B of the third sensor 414B to move the sensing member 418D from the unactuated state to the actuated state.

The processor can therefore determine the direction of rotation of the dial sleeve 406 based on the order of actuation of the sensors 414A, 414B, 414C, 414D. In some embodiments, the determination of the direction of rotation can be made starting from any of the sensors 414A, 414B, 414C, 414D. For example, if the second sensor 414C is actuated, then the processor can determine that the dial sleeve 406 was rotated in the first rotational direction X if the next actuation is of the fourth sensor 414D and can determine that the dial sleeve 406 was rotated in the second rotational direction if the next actuation is of the third sensor 414B.

The processor may be configured to disregard detected rotation of the dial sleeve 406 from the dispensed dosage determination if it is determined that said rotation is in the second rotational direction. For instance, when the dial sleeve 406 rotates in the first rotational direction X during the dispensing of medicament, the dial sleeve 406 will reach the ‘zero’ position once the dial sleeve 406 has been fully rotated in the first rotational direction X and all of the dose has been dispensed. However, it has been found that once the dial sleeve 406 reaches the ‘zero’ position, the dial sleeve 406 may rotate slightly past the ‘zero’ position and then may rotate back again in the second rotational direction (which may be referred to as ‘backspin’), which may cause another one of the sensors 414A, 414B, 414C, 414D to be actuated. If this is further rotation was taken into account in the calculation of the dosage dispensed then the measurement would be higher than the actual dosage delivered. However, since the rotation of the dial sleeve was in the second rotation direction, it can be ignored from the calculation. The dial sleeve 406 may also move in the second rotational direction during rotation of the dose setting dial 408 to ‘dial-in’ the dose.

It should be noted that in an alternative embodiment (not shown), one of the third and fourth sensors 414B, 414D is omitted. In such an arrangement, the processor is still able to determine the direction of rotation of the dial sleeve 406 based on the readings from the three remaining sensors.

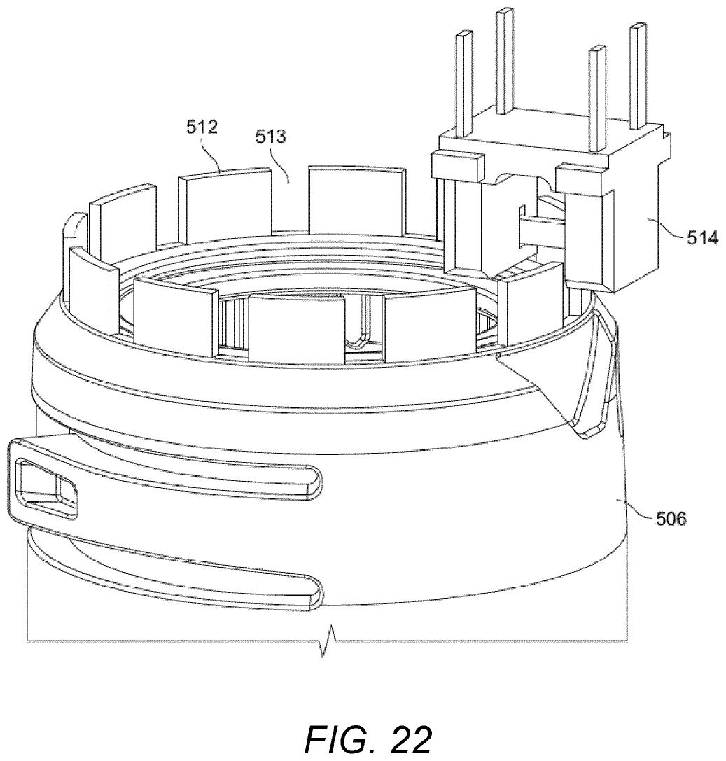

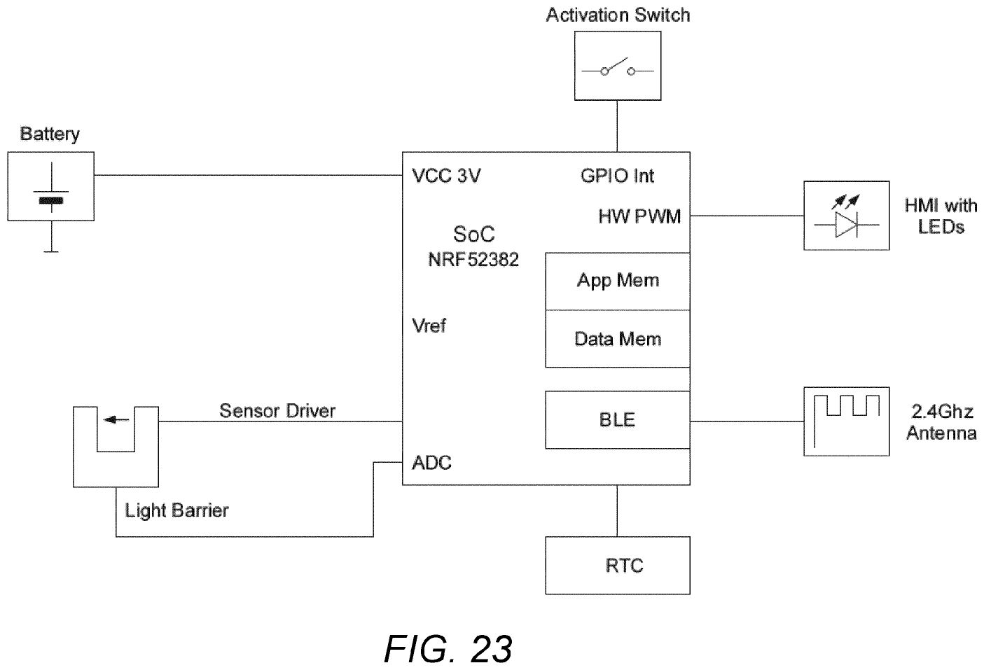

In the above described embodiments, the sensors 114, 214A, 214B, 214C, 214D, 314A, 314B, 314C, 314D, 414A, 414B, 414C, 414D are mechanically actuated. However, it should be recognised that in some embodiments a different type of sensor is used. In an alternative embodiment shown in FIG. 22, the dosage measurement system comprises one or more light sensors 514. The or each lights sensor 514 may comprise a light gate. The dial sleeve 506 may comprise a plurality of formations 512 separated by gaps 513. As the dial sleeve 506 rotates, successive formations 512 align with a light gate to break a light beam of the light gate such that the sensor 514 detects the rotation of the dial sleeve 506. In embodiments with a plurality of sensors 514, the sensors 514 may be arranged as described above in to any of FIG. 16A to FIG. 16C.

In other embodiments (not shown), the sensors 114, 214A, 214B, 214C, 214D, 314A, 314B, 314C, 314D, 414A, 414B, 414C, 414D, 514 may be, for example, magnetic sensors or capacitive sensors. For instance, in one embodiment (not shown) the first encoding regions may comprise a plurality of portions of magnetic material that are adhered to the proximal end of the dial sleeve and are spaced about the rotational axis of the dial sleeve, the second encoding regions being formed as the space between adjacent portions of magnetic material. The or each sensor comprises a magnetic sensor, for example, a Hall sensor, that detects the presence of the magnetic material. In yet another embodiment (not shown), the first encoding regions may comprise a plurality of portions of reflective material that are adhered to the proximal end of the dial sleeve and are spaced about the rotational axis of the dial sleeve, the second encoding regions being formed as the space between adjacent portions of reflective material, or alternatively as portions of material having a different optical property to the first encoding regions, such as being less reflective. The or each sensor comprises an optical sensor that can differentiate between the optical properties of the first and second encoding regions.

In the above described embodiments, the dose setting dial 108 forms an actuator that is moveable axially relative to the housing to operate the dispensing mechanism to dispense medicament from the reservoir. However, in alternative embodiments (not shown) the actuator instead comprises a component that is moveable relative to the dose setting dial to dispense medicament. In one embodiment (not shown), the actuator comprises a push button that is mounted to the dose setting dial and is slidable axially relative to the dose setting dial to operate the dispensing mechanism to dispense medicament from the reservoir. In operation the push button is configured to move axially relative to the housing and dial sleeve by a short distance. This movement occurs when a user exerts a force on the push button. For example, the user pushing push button axially in the distal direction towards the injection site. This axial movement of the push button disengages the clutch such that the clutch moves to a disengaged position and thus allows the dial sleeve and drive sleeve to rotate relative to the housing and dose setting dial in the first rotational direction under the force of the drive member to dispense medicament from the reservoir. The push button may be located at the proximal end of the dose setting dial. Push button may be biased proximally. In some embodiments, the sensors 114, 214A, 214B, 214C, 214D, 314A, 314B, 314C, 314D, 414A, 414B, 414C, 414D, 514 are mounted to the push button to move relative to the housing and dose setting dial. The sensors may move from a rest position to a detecting position upon actuation of the push button, this may also cause an activation switch to be actuated to an on state to power the dosage measurement system, similarly to as described previously. The activation switch may reach the on state prior to the sensor reaching the detecting position.

In some alternative embodiments (not shown), the sensors 114, 214A, 214B, 214C, 214D, 314A, 314B, 314C, 314D, 414A, 414B, 414C, 414D, 514 are not moved axially with the actuator. The sensors may be in a detecting position prior to actuation of the actuator. In some embodiments (not shown), the sensors are fixed relative to the housing.

In the above described embodiments, the encoding regions are provided on the dial sleeve 106, 206, 306, 406, 506 and the or each sensor 114, 214A, 214B, 214C, 214D, 314A, 314B, 314C, 314D, 414A, 414B, 414C, 414D, 514 is configured to detect rotation of the dial sleeve 106, 206, 306, 406, 506. However, in an alternative embodiment (not shown), the encoding regions (for example, the formations and gaps) are provided on the drive sleeve 107 and the or each sensors 114, 214A, 214B, 214C, 214D, 314A, 314B, 314C, 314D, 414A, 414B, 414C, 414D, 514 is configured to detect rotation of the drive sleeve 107. In yet further embodiments (not shown), the encoding regions are provided on another component and the or each sensor 114, 214A, 214B, 214C, 214D, 314A, 314B, 314C, 314D, 414A, 414B, 414C, 414D, 514 is configured to detect rotation of said component. In some embodiments (not shown), the component is a component of the dispensing mechanism.

In some embodiments (not shown), one or more of the signals 219A, 219C, 319A, 319B, 319C, 319D 419A, 419B, 419C, 419D output from the sensors are filtered to improve the accuracy of measurement by, for example, filtering noise from the signals. In some embodiments, the signals are filtered by a low-pass filter. In some embodiments, the signals are filtered before input to the processor. In other embodiments, the processor filters the signals.

Electromechanical switches comprise electrical contacts that may swing for a short time after switching on or off. This may lead to several fast signal pulses after the original signal edge, which is called bouncing. Bouncing can be reduced by switches with oscillation-minimized contact designs. Further, as mentioned above, the signals can be filtered by electronics or software as far as the bouncing duration is shorter than the regular signal pulse duration. This filtering is called debouncing.

It is advantageous to improve power management so that a smaller battery having less capacity can be used, resulting in a smaller dosage measurement system. It is advantageous to reduce peak currents because small batteries are more sensitive to peak currents.

Improved power management may include reducing the energy consumption during shelf-life. In one such embodiment (not shown), the battery is mechanically disconnected during shelf life by a strip, for example, a paper or plastic strip. The strip can be pulled by the user to activate the dosage measurement system. In another embodiment (not shown), the battery is mechanically disconnected by a mechanism. When the actuator is pressed for the first time, a component breaks and enables a durable connection between electronics and battery. In yet another embodiment (not shown), the battery is mechanically disconnected by a life switch on the PCB. This activation may be irreversible. In yet another embodiment, the electronics remains in a strong power save mode until the actuator is pressed for the first time. In yet another embodiment (not shown), the battery is inserted into the device just before first use. In some embodiments (not shown), the button may be replaced or recharged.

The processor may have a minimal sampling interval. In some embodiments, the shortest gaps were around 700 μs. In such embodiments, a sampling interval of 500 μs was enough to detect such short gaps properly. In such an embodiment, a minimum sampling frequency to detect all signal edges is calculated as:

$$f min = \frac{1}{T} \qquad [ Equation 1 ]$$

In Equation 1, fmin is the minimum sampling frequency and T is the sampling interval. Thus, for a sampling interval of 500 μs, the minimum sampling frequency is 2 kHz.

Using mechanical switches instead of light gates/barriers decreases the energy consumption because in some embodiments the states of the mechanical switches can be detected with less than 100 μA per switch. If the signals have a good quality, energy can be saved additionally by detecting the edges per interrupt. This means that the switch may be connected to an interrupt port of the processor. The interrupt port can be selected for being triggered by raising/falling edge in the switch state thereby allowing the processor to wait in a low-energy state instead of performing a permanent energy expensive polling operation at a high sampling frequency.

In embodiments wherein light gates/barriers are used, during the estimated sampling period of 500 μs, the IR-LED may be switched on (rising time typical 10 μs@≤100 kΩ, sinking the same), the ADC may acquire (2 μs) and convert (5 μs) and the rest of the time the IR-LED can be switched off to save power. This has been found to reduce power.

In some embodiments (not shown), the medicament delivery device comprises a user interface, for example, one or more LEDs that prompt or provide indication to the user. The user interface may be optimised to reduce power consumption. For example, if multiple LEDs of one colour are used for a particular prompt or indication, the number of LEDs of that colour may be reduced. In some embodiments, the LED power is reduced. In some embodiments, an injection reminder functionality, for example, a reminder alarm, is removed. In some embodiments, a dwell time indication (for example, a visual or audible indicator of the amount of time that the medicament delivery device should be held in place after injection is removed. In some embodiments, an end of life pattern is removed (for instance, an indicator that the dosage measurement system and/or medicament delivery device has reached the end of its operational life. In some embodiments, the user interface is omitted.

In some embodiments, a BLE (Bluetooth™ Low Energy) advertising duration is reduced. The advertising time defines how long the medicament delivery device tries to connect to the mobile phone or other device. This parameter is important when the mobile phone is often unreachable (for example, being too far away or switched off). Experiments have shown that 5 seconds are enough when the mobile phone is inside the properly reachable radio-range. So the advertising time could be reduced from 15 seconds to around 5 seconds. This means that the advertising energy can be improved by a factor of 3.

In some embodiments, the BLE communication timeout is reduced. After the data transfer the communication channel stays currently open for 3 seconds. This can be improved when the mobile phone or other device sends an explicit communication close command to the medicament delivery device and the timeout is just left as fall-back.

In some embodiments, the number of communication attempts per day is reduced. The data could be transferred to the mobile phone after an injection, but only when the last data transfer was more than a predetermined time period (for example, 12 hours) prior to that. In some embodiments, priming log data is never transferred separately. This helps to save energy for users with several injections and/or priming doses per day.

There might be use cases where users want to force an immediate data transfer. This could be initiated by a button press pattern. One step more would be to transmit only after a specific button press pattern (for example, pressing the actuator or another button five times in quick succession, or a certain number of long presses of the button). In some embodiments, data is transmitted after a normal button release event, but only if last data transfer was more than a predetermined time period (for example, 12 hours) prior to that. In some embodiments, priming data is not transmitted separately. For example, if the user performs a priming operation in which one or two increments of rotation are detected by the processor, then this is not transmitted separately to the medicament delivery data (in some embodiments, primings, for example, measurements equal to or less than two increments of rotation, are disregarded and not transmitted).

It is advantageous to avoid or reduce current peaks because they reduce the usable capacity of batteries. It is therefore advantageous to optimise the energy management for the UI, the sensor and the communication current peaks. In some embodiments, current peaks of electronic components are aligned successively. In some embodiments, components with high currents are not active at the same time as components with high peak currents, or at least such simultaneous occurrences are minimised.

The visible LED may have high peak currents, but the timing is controllable. The BLE communication may have high peak currents, which may not be controllable nor predictable. Therefore, in some embodiments communication is only started after a potential user feedback by LED has completed.

The radio range may be dependent on the transmit power of the dosage measurement system (for example, the System on Chip, SoC, component). Reducing the power will reduce the average current and the peak current. In some embodiments, the maximum output power is +0 dBm and has a peak current of 5.3 mA. If the maximum output power was instead+4 dBm then the peak current would be 7.5 mA, which may be too high for a smaller battery type. Reducing the output power to −4 dBm results in a peak current of 4.2 mA.

In some embodiments, the battery is a CR1225 coin cell having a nominal capacity of 48 mAh.

While the embodiments above have been described in relation to collecting data from an insulin injector pen, it is noted that embodiments of the invention may be used for other purposes, such as monitoring of injections of other medicaments.

The injection device may comprise a cartridge containing a liquid drug or medicament. In instances, by pressing the injection button a portion thereof may be expelled from the cartridge according to the dialed or pre-set amount. The terms “drug” and “medicament”, may refer to a pharmaceutical formulation containing at least one pharmaceutically active compound. More details on particular pharmaceutical formulations may be taken from the disclosure of the co-pending application PCT/EP2018/082640, attorney docket DE2017/081, which, to this extent, shall be included herein by reference.

Those of skill in the art will understand that modifications (additions and/or removals) of various components of the substances, formulations, apparatuses, methods, systems and embodiments described herein may be made without departing from the full scope and spirit of the present invention, which encompass such modifications and any and all equivalents thereof.

1.-36. (canceled)

37. A medicament delivery device comprising:
a reservoir for medicament;
a dispensing mechanism operable to dispense medicament from the reservoir, the dispensing mechanism comprising a sleeve configured to rotate during the dispensing of medicament and having a plurality of formations at an end of the sleeve; and
a dosage measurement system comprising (i) at least one mechanically actuated sensor configured such that rotation of the sleeve causes successive formations to engage the sensor such that the sensor detects rotation of the sleeve and (ii) a processor configured to determine a dosage dispensed from the medicament reservoir based on the detected rotation of the sleeve.

38. The medicament delivery device according to claim 37, wherein the plurality of formations comprise a plurality of teeth.

39. The medicament delivery device according to claim 37, wherein the plurality of formations are formed on a proximal end of the sleeve.

40. The medicament delivery device according to claim 37, wherein the sleeve is a dial sleeve or a drive sleeve.

41. The medicament delivery device according to claim 37, wherein the sensor comprises a sensing member that is configured to move from an unactuated state to an actuated state when the sensing member is engaged by one of the plurality of formations during rotation of the sleeve, wherein the sensor detects movement of the sensing member between the unactuated and actuated states.

42. The medicament delivery device according to claim 41, wherein each formation comprises a leading edge and wherein engagement of the sensor with the leading edge of a first formation of the plurality of formations until engagement of the sensor with the leading edge of an adjacent second formation of the plurality of formations during rotation of the sleeve represents one encoding period, and wherein the sensor is actuated for between 40% to 60% of the encoding period.

43. The medicament delivery device according to claim 37, comprising a dose dial and a housing, wherein the dose dial is configured to be rotated relative to the housing to set a dose of medicament to be delivered by the dispensing mechanism, and wherein the sensor is mounted to the dose dial.

44. The medicament delivery device according to claim 43, comprising a torque limiter, wherein the sensor is mounted to the dose dial via the torque limiter such that rotation of the dose dial relative to the housing with a torque greater than a predetermined limit causes the torque limiter to move to an open state such that the dose dial can rotate relative to the sensor.

45. The medicament delivery device according to claim 43, comprising a one-way mechanism, wherein the sensor is mounted to the dose dial via the one-way mechanism such that the sensor is resisted from rotating relative to the dose dial in the direction that the sleeve rotates during the dispensing of medicament and is permitted to rotate relative to the dose dial in the opposite direction that the sleeve rotates during the dispensing of medicament.

46. A dosage measurement system for a medicament delivery device, wherein the medicament delivery device comprises: a housing containing a reservoir for medicament; and a dispensing mechanism operable to dispense medicament from the reservoir and comprising a component configured to rotate during the dispensing of medicament, the component comprising a plurality of formations; and an actuator configured to be movable relative to the housing upon actuation to operate the dispensing mechanism to dispense medicament from the reservoir, the dosage measurement system comprising:
a sensor moveable from an idle position to a detecting position wherein rotation of the component causes successive formations to be detected by the sensor such that the sensor detects rotation of the component;
a processor configured to determine a dosage dispensed from the medicament reservoir based on the detected rotation of the component; and
an activation switch moveable from an initial off state to an on state to activate the dosage measurement system upon actuation of the actuator, the sensor configured to be mounted to the actuator such that actuation of the actuator causes the sensor to move relative to the housing to reach the detecting position, wherein the activation switch reaches the on state prior to the sensor reaching the detecting position.

47. The dosage measurement system according to claim 46, wherein the actuator is slidable relative to the housing.

48. The dosage measurement system according to claim 46, wherein the activation switch comprises a pivotal member that pivots from the off state to the on state to activate the dosage measurement system.

49. The dosage measurement system according to claim 46, wherein the medicament delivery device comprises a stop and wherein the activation switch is configured to rest against the stop when the activation switch is in the off state.

50. The dosage measurement system according to claim 46, wherein the activation switch is configured to engage a part of the medicament delivery device when the actuator is moved to operate the dispensing mechanism such that the activation switch is urged to the on state.

51. The dosage measurement system according to claim 46, wherein the activation switch is configured such that the activation switch moves a first distance from the off state to the on state and the sensor is configured such that the sensor moves a second distance from the idle position to the detecting position, wherein the second distance is greater than the first distance.

52. The dosage measurement system according to claim 46, comprising first and second radial bearings, wherein the actuator is rotatably mounted to a part of the device via the first and second bearings, the first and second bearings being axially spaced.

53. The dosage measurement system according to claim 46, comprising a support member and a coupling member, wherein the actuator is fixed relative to the support member and wherein the support member is coupled to the coupling member.

54. A dosage measurement system for a medicament delivery device, wherein the medicament delivery device comprises a reservoir for medicament and a dispensing mechanism operable to dispense medicament from the reservoir, the dispensing mechanism comprising a component configured to rotate during the dispensing of medicament, the component comprising a plurality of first and second encoder regions, the dosage measurement system comprising:
first and second sensors that are offset such that rotation of the component causes one of the first encoder regions to align with the first sensor whilst one of the second encoder regions aligns with the second sensor and then one of the second encoder regions, to align with the first sensor whilst one of the first encoder regions aligns with the second sensor, the first and second sensors configured to differentiate between the first and second encoder regions to detect rotation of the component; and
a processor configured to determine a dosage dispensed from the medicament reservoir based on the detected rotation of the component.

55. The dosage measurement system according to claim 54, wherein the component comprises a plurality of formations, wherein each first encoding region comprises at least a portion of a respective formation that is detectable by the first and second sensors as the component rotates, wherein the second encoder regions are provided between adjacent first encoder regions.

56. The dosage measurement system according to claim 54, wherein each formation comprises a tooth, wherein each first encoding region comprises at least a portion of a respective tooth that is detectable by the first and second sensors as the component rotates, wherein each second encoder region comprises at least a gap between adjacent teeth.

57. The dosage measurement system according to claim 54, wherein the first and second sensors are arranged such that for all rotational positions of the component in which the first sensor aligns with one of the first encoding regions, the second sensor aligns with one of the second encoding regions.

58. The dosage measurement system according to claim 54, wherein the component comprises a plurality of encoding periods, wherein each encoding period comprises one of the first encoding regions and an adjacent second encoding region, wherein for a given rotational position of the component the first sensor is aligned with a portion of one of the encoding periods and the second sensor is aligned with a different portion of one of the encoding periods.

59. The dosage measurement system according to claim 58, further comprising a third sensor that is aligned with a different portion of one of the encoding periods to the first and second sensors when the component is in said given rotational positon.

60. The dosage measurement system according to claim 59, further comprising a fourth sensor that aligns with a different portion of one of the encoding periods to the first, second and third sensors when the component is in said given rotational positon.

61. The dosage measurement system according to claim 54, further comprising a third sensor configured such that, in use, when the component is rotated the third sensor aligns with one of the first encoding regions at the same time that the first sensor aligns with one of the first encoding regions.

62. The dosage measurement system according to claim 61, further comprising a fourth sensor configured such that when the component is rotated the fourth sensor aligns with one of the first encoding regions at the same time that the second sensor aligns with one of the first encoding regions.

63. The dosage measurement system according to claim 54, wherein each of the first and second encoding regions extends about the rotational axis of the component by the same predetermined angle.

64. The dosage measurement system according to claim 63, wherein the second sensor is offset from the first sensor about the rotational axis in a first direction by an odd number integer multiple of the angle that each first encoding region subtends about the rotational axis.

65. The dosage measurement system according to claim 54, wherein the processor is configured to determine a dosage dispensed from the medicament reservoir by a process including counting the number of transitions between the first and second encoding regions detected by the first and second sensors.

66. The dosage measurement system according to claim 54, wherein the first and second sensors are configured to move from an idle position to a detecting position, wherein movement of the sensors to the detecting position causes one of the first and second sensors to align with one of the first encoding regions, and wherein the processor is configured such that when determining the dosage dispensed the processor compensates for said alignment when the sensor moves to the detecting positon.

67. The dosage measurement system according to claim 54, wherein the processor is configured to determine a dosage dispensed from the medicament reservoir based on a signal from one of the first and second sensors and an inverse signal from the other one of the first and second sensors.

68. The dosage measurement system according to claim 67, wherein the processor is configured to determine a dosage dispensed from the medicament reservoir based on a superposition of the signal from one of the first and second sensors and the inverse signal from the other one of the first and second sensors.

69. The dosage measurement system according to claim 68, wherein the processor is configured to determine a dosage dispensed from the medicament reservoir by comparing the superposition to a first threshold value and a second threshold value greater than the first threshold value.
